(19) United States
(12) Patent Application Publication
(10) Pub. No.: US 2025/0160866 A1
(43) Pub. Date: May 22, 2025

(54) SUPER-BORE CATHETER WITH BRAID SUPPORTED FLARED TIP

(71) Applicant: Neuravi Limited, Galway (IE)

(72) Inventors: Karl KEATING, Galway (IE); David VALE, Barna (IE); Declan LEE, Longford (IE); Sinead KENNY, Galway (IE); Michael SCANLON, Galway (IE); Michael GILVARRY, Headford (IE)

(73) Assignee: Neuravi Limited, Galway (IE)

(21) Appl. No.: 18/912,912

(22) Filed: Oct. 11, 2024

Related U.S. Application Data
(60) Provisional application No. 63/599,805, filed on Nov. 16, 2023.

(51) Int. Cl. A61B 17/221; A61B 17/22; A61M 25/00; A61M 25/01

(52) U.S. Cl. CPC A61B 17/221; A61B 2017/22079; A61B 2017/2215; A61M 25/0074; A61M 25/0108

(57) ABSTRACT

The designs and methods disclosed herein are for a clot retrieval catheter with a large bore shaft and a distal braid-supported tip that is expandable to a diameter larger than the outer sheath through which it is delivered.

### CROSS REFERENCE TO RELATED APPLICATION

The present application claims benefit of priority to U.S. Provisional Patent Application No. 63/599,805 filed Nov. 16, 2023. The entire contents of which are hereby incorporated by reference.

### FIELD OF INVENTION

The present invention generally relates devices and methods for removing acute blockages from blood vessels during intravascular medical treatments. More specifically, the present invention relates to retrieval catheters with expandable tips into which an object or objects can be retrieved.

### BACKGROUND

Clot retrieval aspiration catheters and devices are used in mechanical thrombectomy for endovascular intervention, often in cases where patients are suffering from conditions such as acute ischemic stroke (AIS), myocardial infarction (MI), and pulmonary embolism (PE). Accessing the neurovascular bed in particular is challenging with conventional technology, as the target vessels are small in diameter, remote relative to the site of insertion, and highly tortuous. These catheters are frequently of great length and must follow the configuration of the blood vessels in respect of all branching and windings. Traditional devices are often either too large in profile, lack the deliverability and flexibility needed to navigate particularly tortuous vessels, or are not effective at removing a clot when delivered to the target site.

Many existing designs for aspiration retrieval catheters are often restricted to, for example, inner diameters of 6Fr or between approximately 0.068-0.074 inches. Larger sizes require a larger guide or sheath to be used, which then necessitates a larger femoral access hole to close. Most physicians would prefer to use an 8Fr guide/6Fr sheath combination, and few would be comfortable going beyond a 9Fr guide/7Fr sheath combination. This means that once at the target site, a clot can often be larger in size than the inner diameter of the aspiration catheter and must otherwise be immediately compressed to enter the catheter mouth. This compression can lead to bunching up and subsequent shearing of the clot during retrieval. Firm, fibrin-rich clots can also become lodged in the fixed-mouth tip of these catheters making them more difficult to extract. This lodging can also result in shearing where softer portions break away from firmer regions of the clot.

Small diameters and fixed tip sizes are also less efficient at directing the aspiration necessary to remove blood and thrombus material during the procedure. The suction must be strong enough such that any fragmentation that may occur as a result of aspiration or the use of a mechanical thrombectomy device cannot migrate and occlude distal vessels. When aspirating with a fixed-mouth catheter, a significant portion of the aspiration flow ends up coming from vessel fluid proximal to the tip of the catheter, where there is no clot. This significantly reduces aspiration efficiency, lowering the success rate of clot removal.

Large bore intermediate and aspiration catheters and/or those with expandable tips are therefore desirable because they provide a large lumen and distal mouth to accept a clot with minimal resistance. The bore lumen of these catheters can be nearly as large as the guide and/or sheath through which they are delivered, and the expandable tip can expand to be a larger diameter still. When a clot is captured and drawn proximally into a tip with a funnel shape, the clot can be progressively compressed during retrieval so that it can be aspirated fully through the catheter and into a syringe or cannister. Funnel shaped catheters also lend themselves to better alignment in a vessel, as when the size of the tip is close to that of a vessel the device can self-center. The smaller diameter just proximal of the funnel portion of the tip can allow a hinging motion for bending between the tip and the shaft, in contrast to fixed-mouth designs where there is less freedom of motion and the tip and shaft will tend to move together.

In many examples, the fixed-mouth catheters and those with expandable tips can have an underlying braid as the primary supporting backbone. The use of braids in a catheter body is not a novel concept, and typical examples can be readily found in the art. The braid can often be as simple as bands wrapped spirally in one direction for the length of the catheter which cross over and under bands spiraled in the opposite direction. The bands can be metallic, fiberglass, or other material providing effective hoop strength to reinforce the softer outer materials of the body. However, supporting braids can often lack an effective bonding mechanism for the layers, or have a high sectional stiffness to the point where they do not meet the flexibility criteria for many procedures. Additionally, many of these devices have structures which cannot be made soft enough for use in fragile vessels without causing substantial trauma.

Combining the clinical needs of these catheters without significant tradeoffs can be tricky. Catheter designs attempting to overcome the above-mentioned design challenges would need to have a large bore and an expandable tip with sufficient hoop strength in the expanded state to resist aspiration forces without collapse while having a structure capable of folding down consistently and repeatably when retrieved into an outer guide and/or sheath. The tip structure needs to have the flexibility and elasticity to survive the severe mechanical strains imparted when navigating the tortuous vasculature, while also being capable of expanding elastically as a clot is ingested for better interaction with and retention of the clot.

Axially, the tip shape must maintain good pushability so that it can be advanced within an outer sheath, and in the expanded state in a vessel with minimal tendency to further over-expand outward when placed in compression. The tip in the expanded state can also be advanced through vessels that are smaller in diameter, as the funnel shape allows the tip to radially compress when advanced through a narrowing vessel with minimal force. This is advantageous as the tip can seal in a wider range of vessel sizes.

As a result, there remains a need for improved catheter designs attempting to overcome the above-mentioned design challenges. The presently disclosed designs are aimed at providing an improved retrieval catheter with an expansile tip and methods for fabricating such a catheter capable of improved performance.

### SUMMARY

It is an object of the present designs to provide devices and methods to meet the above-stated needs. The designs can be for a clot retrieval catheter capable of remove a clot from cerebral arteries in patients suffering AIS, from coronary native or graft vessels in patients suffering from MI, and from pulmonary arteries in patients suffering from PE and from other peripheral arterial and venous vessels in which a clot is causing an occlusion. The designs can also resolve the challenges of aspirating fibrin rich clot material by addressing the key difficulties of 1) the friction between the clot and the catheter and 2) the energy/work required to deform these firm clots as they are aspirated into the catheter tip.

Certain implementations of the present disclosure provide a catheter. The catheter includes a proximal elongate shaft including a distal end, a lumen, and a first plurality of wire braided segments. The catheter includes a distal tip section connected to the distal end of the proximal elongate shaft. The distal tip section includes (i) a proximal tubular body, (ii) an expansile portion having a collapsed delivery configuration and an expanded deployed configuration, and (iii) a second plurality of wire braided segments including circumferential rings of cells. The distal end of the expansile portion includes distal hoops where wires of the second plurality of wire braided segments invert to loop back through the second plurality of wire braided segments and create distalmost cells of the expansile portion. The catheter includes one or more polymer body jackets disposed around the proximal elongate shaft and one or more polymer distal jackets disposed around the distal tip section. The catheter includes one or more radiopaque pucks positioned within one or more of the distalmost cells.

Another aspect of the present disclosure provides a catheter. The catheter includes a proximal elongate shaft includes a distal end, a lumen, and a first plurality of wire braided segments. The catheter includes a distal tip section connected to the distal end of the proximal elongate shaft. The distal tip section includes (i) a proximal tubular body, (ii) an expansile portion having a collapsed delivery configuration and an expanded deployed configuration, and (iii) a second plurality of wire braided segments including circumferential rings of cells. The catheter includes a plurality of polymer jackets extending from the proximal elongate shaft to proximate the distal end of the expansile portion. The plurality of polymer jackets include a first polymer jacket and a second polymer jacket. The catheter includes a transition jacket positioned proximate a transition between the first plurality of wire braided segments and the second plurality of wire braided segments. The transition jacket has a hardness greater than a hardness of either of the first polymer jacket proximal to the transition and the second polymer jacket distal to the transition.

Another aspect of the present disclosure provides a catheter. The catheter includes a proximal elongate shaft including a distal end, a lumen, and a first plurality of wire braided segments. The catheter includes a distal tip section connected to the distal end of the proximal elongate shaft, the distal tip section including (i) a proximal tubular body, (ii) an expansile portion having a collapsed delivery configuration and an expanded deployed configuration, and (iii) a second plurality of wire braided segments including circumferential rings of cells. The catheter includes one or more polymer body jackets disposed around the proximal elongate shaft and one or more polymer distal jackets disposed around the distal tip section. A distal end of the expansile portion includes distal hoops where wires of the second plurality of wire braided segments invert to loop back through the second plurality of wire braided segments. The expansile portion includes at least a first circumferential rings of cells proximate a transition between the proximal tubular body and the expansile portion, a second circumferential rings of cells adjacent and distal to the first circumferential rings of cells, and a third circumferential rings of cells proximate a distal end of the expansile portion. The first circumferential rings of cells includes closed cells having a first braid angle, the second circumferential rings of cells includes closed cells having a second braid angle, and the third circumferential rings of cells includes closed cells having a third braid angle. The first braid angle is greater than or equal to the second braid angle minus 5°, the first braid angle is approximately 65°, and the third braid angle is approximately 53°.

Another aspect of the present disclosure provides an insertion tool. The insertion tool includes an elongated shaft with an inner lumen. The elongated shaft includes a distal shaft section, a proximal flare section having an inner diameter greater than that of the distal shaft section, and a distal tip having a tapered portion.

Other aspects of the present disclosure will become apparent upon reviewing the following detailed description in conjunction with the accompanying figures. Additional features or manufacturing and use steps can be included as would be appreciated and understood by a person of ordinary skill in the art.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation. It is expected that those of skill in the art can conceive of and combine elements from multiple figures to better suit the needs of the user.

FIG. 1 is a view of a large bore clot retrieval catheter with an expandable tip being advanced through the vasculature, according to aspects of the present invention;

FIG. 2 illustrates the distal portion of a large bore clot retrieval catheter with an expandable tip, according to aspects of the present invention;

FIG. 3 shows a cutaway view of a large bore clot retrieval catheter, according to aspects of the present invention;

FIG. 4 depicts the proximal end of the large bore clot retrieval catheter of FIG. 3, according to aspects of the present invention;

FIG. 5 shows the distal end of the large bore clot retrieval catheter of FIG. 3, according to aspects of the present invention;

FIG. 6A illustrates an example braid configuration for the distal tip section, according to aspects of the present invention;

FIG. 6B is a side view of a catheter showing transitions from the proximal elongate shaft to the distal tip section, according to aspects of the present invention;

FIG. 6C is a detailed view of the area circled proximally in FIG. 6B and shows a transition from the proximal elongate shaft to the distal tip section, according to aspects of the present invention;

FIG. 7 is another example of a braid configuration for the distal tip section, according to aspects of the present invention;

FIG. 8 is a view of an expanded distal tip section with a disk extension, according to aspects of the present invention;

FIGS. 9A-9K illustrate steps of a method for the construction of a catheter, according to aspects of the present invention;

FIG. 10A is a detailed view of the area circled distally in FIG. 6B and shows a distal tip section of a catheter, according to aspects of the present invention;

FIG. 10B is a view of an alternative design for a distal tip section of a catheter to the one shown in FIG. 10A, according to aspects of the present invention;

FIG. 11 is a detailed view of the area circled in FIG. 10B and shows a distal tip section of a catheter with a distal overlap jacket, according to aspects of the present invention;

FIG. 12 illustrates a catheter attached to a connection hub, according to aspects of the present invention; and

FIGS. 13A-13D illustrate an example insertion tool, with FIG. 13A providing a side view, FIG. 13B providing an end view, FIG. 13C providing a top elevational view, and FIG. 13D providing a detail view of a distal tip of the insertion tool, according to aspects of the present invention.

### DETAILED DESCRIPTION

Specific examples of the present invention are now described in detail with reference to the Figures, where identical reference numbers indicate elements which are functionally similar or identical. The examples address many of the deficiencies associated with traditional clot retrieval aspiration catheters, such as poor or inaccurate deployment to a target site and ineffective clot removal.

The designs herein can be for a super-bore clot retrieval catheter with a large internal lumen and a distal funnel tip that can self-expand to a diameter larger than that of the guide or sheath through which it is coaxially delivered. The designs can have a proximal elongate body for the shaft of the catheter, and a distal tip with an expanding braided support structure and outer polymeric jacket to give the tip atraumatic properties. The braided support can be designed so that the expansion capability is variably focused in an axial portion of the tip section. The braid cells can be capable of easily and repeatably collapsing for delivery and expanding for good clot reception and resistance under aspiration. Sections of the tip can have the ability to further expand beyond the free shape of the expanded deployed configuration when ingesting a clot. The catheter's braid and tip designs can be sufficiently flexible to navigate highly tortuous areas of the anatomy and be able to recover its shape to maintain the inner diameter of the lumen when displaced in a vessel.

Accessing the various vessels within the vascular, whether they are coronary, pulmonary, or cerebral, involves well-known procedural steps and the use of a number of conventional, commercially-available accessory products. These products, such as angiographic materials, mechanical thrombectomy devices, microcatheters, and guidewires are widely used in laboratory and medical procedures. When these products are employed in conjunction with the devices and methods of this invention in the description below, their function and exact constitution are not described in detail. Additionally, while the description is in many cases in the context of thrombectomy treatments in intercranial arteries, the disclosure may be adapted for other procedures and in other body passageways as well.

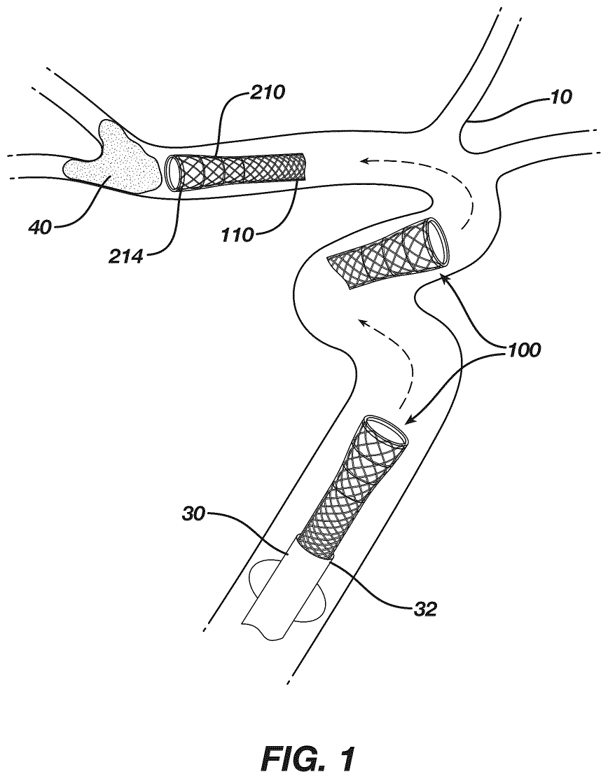

Turning to the figures, FIG. 1 illustrates a possible sequence for approaching an occlusive clot 40 using a large bore clot retrieval catheter 100 of the designs disclosed herein. The clot 40 can be approached with the catheter 100 collapsed within a guide sheath 30 or other outer catheter for delivery. When the vasculature 10 becomes too narrow and/or tortuous for further distal navigation with the guide sheath 30, the catheter 100 can be deployed for further independent travel distally. The catheter 100 can be highly flexible such that it is capable of navigating the M1 or other tortuous regions of the neurovascular to reach an occlusive clot.

The clot retrieval catheter 100 can have a flexible elongate shaft 110 serving as a shaft with a large internal bore (which in some cases can be 0.090 inches or larger) and a distal tip section 210 having a collapsible supporting braided structure. The large bore helps the catheter to be delivered to a target site by a variety of methods. These can include over a guidewire, over a microcatheter, with a dilator/access tool, or by itself.

In most cases, the design of the collapsible funnel tip can be configured so that the catheter 100 can be delivered through (and retrieved back through) commonly sized outer sheaths and guides. For example, a standard 6Fr sheath/8Fr guide, would typically have an inner lumen of less than 0.090 inches. The tip can then be designed with a collapsed delivery outer diameter of approximately 0.086 inches. The tip can self-expand once advanced to an unconstrained position distal to the distal end 32 of the guide sheath 30, capable of reaching expanded outer diameters as large as approximately 0.132 inches. As the catheter can be delivered independently to a remote occlusion, the tip section 210 must be designed to be able to resist collapse from the forces of aspiration, have excellent lateral flexibility in both the expanded and collapsed states, an atraumatic profile to prevent snagging on bifurcations in vessels, and conformability to allow self sizing should the tip need to be advanced through vessels with a diameter smaller than the tip and track past calcified plaque without dislodging it.

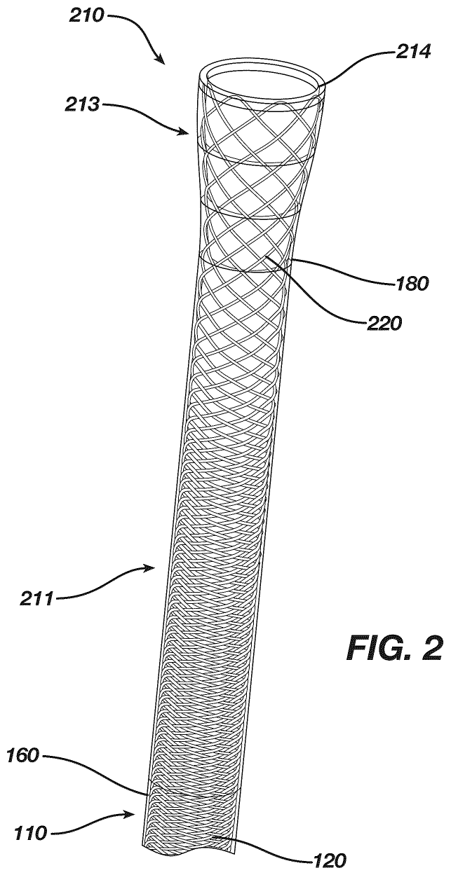

A closer view of the distal portion of the catheter with the tip section 210 in the expanded deployed configuration as a funnel is illustrated in FIG. 2. The elongate shaft 110 can have a backbone consisting of a first plurality of braid sections 120 enclosed by an axial series of outer body jackets 160. As used herein, “braided sections” can refer to segments within a single monolithic braid that have different physical properties and/or configurations and does not necessarily mean two distinct structures bonded together.

Similarly, the tip section 210 can have another series of braided segments 220 surrounded by one or more polymer distal jackets 180 near the expansile portion 213. It can be appreciated that different braided sections of the tip and shaft braids can have different geometries and weave patterns to achieve desired properties for that segment of catheter. It is also appreciated that the tip section braid can extend for the full length of the catheter so that a separate proximal braid is not required.

The tip section 210 can have a first substantially tubular proximal body 211 and a second distal expansile portion 213. Changes to the braid properties can be more pronounced in the expansile portion 213 to allow for radial expansion when the catheter is deployed largely funnel shape and, in some cases, additional expansion when a large, stiff clot is swallowed into the distal mouth of the tip. The length and contour of the funnel portion of the expansile portion 213, as tapering from the distal end 214, can be also adjusted by through heat set of the braid wires by designing the elastic free shape. A short funnel can maintain good hoop stiffness and flexibility through having a shorter lever distance to hinge off the elongate shaft 110. A short funnel can also be tailored to minimize stretch and deformation in the more distal of the polymer distal jackets 180. Alternatively, a longer funnel with a shallower taper can better interact with and more gradually compress a clot over the length to reduce the risk of lodging.

The tip shape and polymer distal jackets 180 can block some of the proximal fluid from entering the expanded tip during aspiration and retrieval of the clot, allowing for more efficient direction of the aspiration force and prevention of the distal migration of clot fragments or other debris during the procedure. The polymer distal jackets 180 can be formed from a highly-elastic material such that the radial force exerted by expanding the expansile tip is sufficient to stretch the membrane to the funnel shape contours of the tip when in the expanded deployed configuration. One example can be using a ductile elastomer which has the advantages of being soft and flexible with resistance to tearing and perforation due to a high failure strain. Alternately, the jacket can be baggy and loose and fold over the support frame edges so that the frame can move more freely when expanded and collapsed.

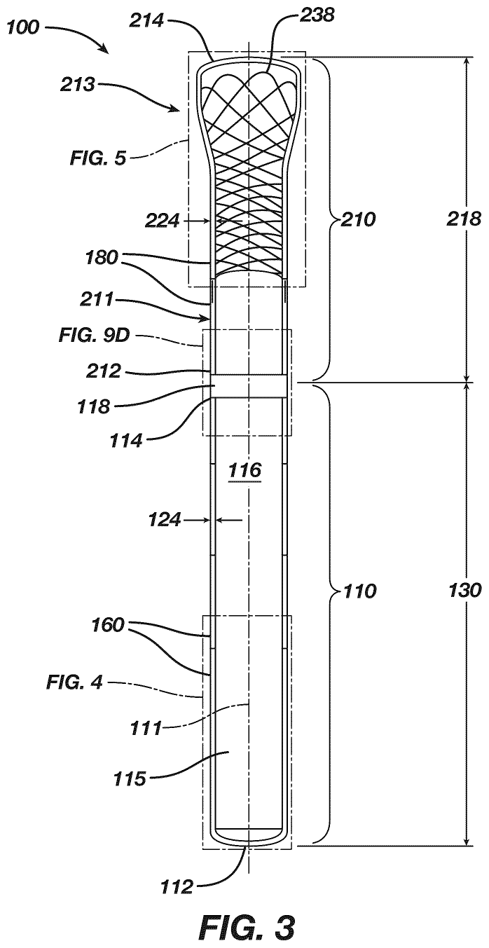

FIG. 3 shows a cutaway view of a design for an aspirating clot retrieval catheter 100. The catheter 100 can have a proximal tubular elongate shaft 110 configured around a longitudinal axis 111. The elongate shaft 110 can have an underlying braided support structure (not shown for clarity) defining the catheter lumen 116, which can have a low friction inner liner 115 and be used for the delivery of auxiliary devices, contrast injection, and direct distal aspiration to a clot face. Preferred liner materials can be polytetrafluoroethylene (PTFE), wrapped ePTFE, or similar materials. A distal tip section 210 can have a self-expandable expansile portion 213 that is effectively spring-loaded within the outer sheath when collapsed for delivery. The shaft 110 and tip section 210 can be concentric with the longitudinal axis 111 and share the same approximate diameter for smooth delivery through an outer sheath.

The outer surface of the elongate shaft 110 can be a series of five to eight, e.g., six or seven, outer polymer body jackets 160. The body jackets 160 can be made of various medical grade polymers, such as Chronoprene, Neusoft, Engage, polyether block amide (Pebax®), or Nylon. Materials can be chosen, for example, so that progressively more proximal segments are generally harder and less flexible (by durometer hardness, flexure modulus, etc.) for pushability as the proximal end of the catheter is approached. The polymer body jackets 160 can be reflowed over the underlying braid and allowed to flow through the interstitial spaces of the braid cells to bond the layers of the construct together. The polymer body jackets 160 can be butted together to form a continuous and smooth outer surface for the catheter shaft. As will be described in greater detail below, the hardness of the polymer body jackets 160 can decrease from proximal to distal along the length of the catheter 100, wherein the hardest jackets are proximal and the softer jackets are distal.

The distal tip section 210 can be subdivided into two or more regions, depending on the expansion characteristics allowed by the underlying braid. A proximal tubular body 211 can have largely the same profile and diameters as the elongate shaft 110 and transition into a distal expansile portion 213 designed to passively expand when deployed from the outer sheath and/or guide. The tip section can have one or more polymer distal jackets 180, with the hardness of the jackets becoming progressively less as the distal end 214 of the section is approached to give the catheter a soft, atraumatic end.

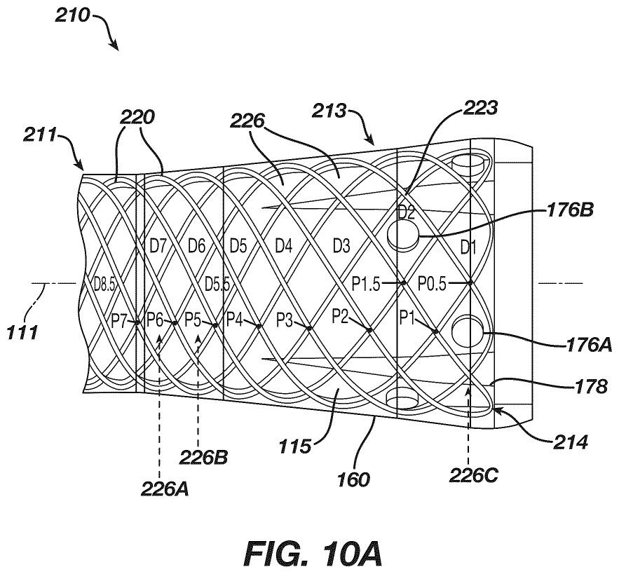

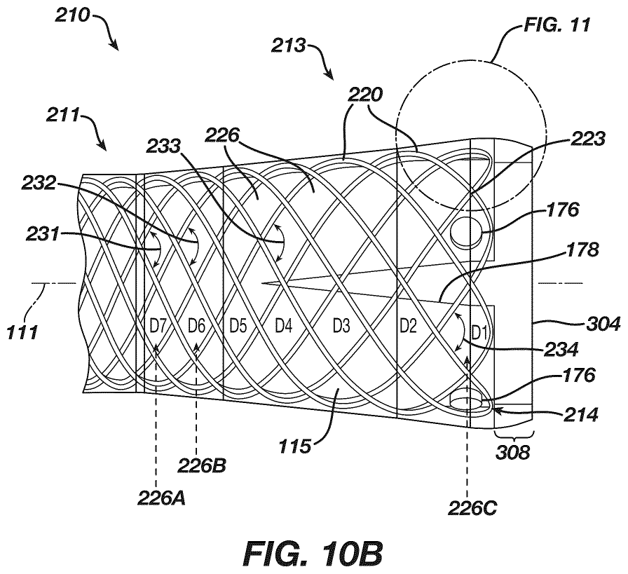

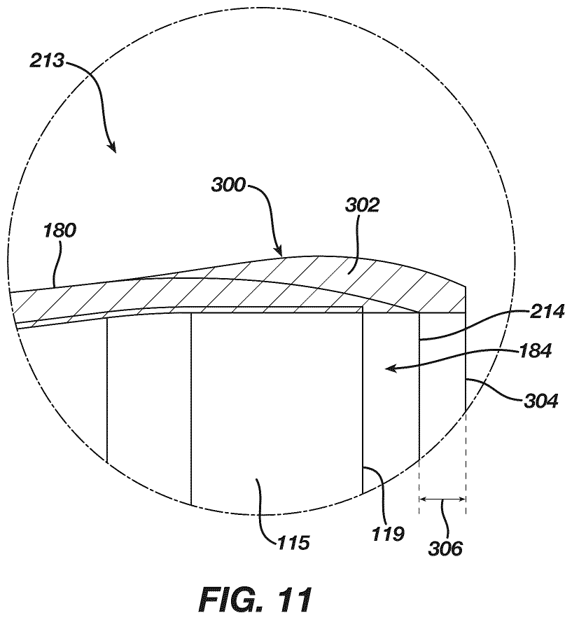

The wires of the underlying braid can be wound in one direction from the proximal end 212 of the tip section distally. Upon reaching a distal terminus, the wires can be inverted 238 to form distal hoops 230 and wind proximally in the opposite direction. As a result, the inverted ends are also more atraumatic as the free ends of the wires exist only at the proximal end 212 of the braid. This contrasts with, for example, other catheter designs where the braided reinforcing sections resemble a stent with wire free ends existing on both opposing ends of the device. The braid designs disclosed herein have the additional advantage of ensuring the sum of wires wrapped in one direction will necessarily be equal to those wound in the opposite direction, preventing any helical curl which could otherwise exist in the catheter as a result of any imbalance. The hoops 230 define the distalmost cells 228 of the expansile portion 213. The location of these distalmost cells 228 provide a preferred distal location for radiopaque markers, such as the pucks 176 described in greater detail below with reference to FIGS. 10A and 11.

Differing braid patterns for the elongate shaft 110 and tip section 210 provide specific mechanical properties to the finished device. Some patterns can offer kink resistance and burst strength but can lack pushability. Other patterns offer greater torqueability and scaffolding support for the outer jackets at the cost of some flexibility. Still other patterns can give excellent flexibility and energy dispersion.

Additionally, other factors can be tuned as well. The catheter does not necessarily have to have two discrete braided sections for the shaft and distal tip. One three, four, or more discrete sections of differing flexibility can be used. Choices for other physical parameters, such as wall thickness and material composition, can also be implemented for the various catheter sections. For example, the stiffer and more proximal polymer body jackets 160 can have a wall thickness 124 of approximately 0.004 inches to maintain column stiffness in the longitudinal direction without compromising much lateral flexibility. Ideally, there will be at least 0.00025″, and more preferably 0.0005″ to 0.0015″, of jacket material residing above the braid wires to ensure braid wires are fully encapsulated, with the inner surface of the braid being in contact with or residing no more than 0.002″, and more preferrable 0.0005″, in diameter above the liner. More expansile tip jackets, or polymer distal jackets 180, can have a slightly increased wall thickness 224 of approximately 0.006 to give the expandable tip an additional atraumatic cushion where very soft jacket materials are used. The thicker wall also allows for material to reside both below and above the braid wires for full encapsulation where no liner is used. Liners are best avoided for the tip section as standard liners are not elastomeric and may plastically deform through expansion and compression. It is appreciated that an elastomeric linear could be used for the tip section that is of a different hardness to the outer jacket of the tip section.

Radiopaque marker bands can be included at different axial points along the length of the catheter 100 for visibility under fluoroscopy during a procedure. In the example illustrated, a marker band 118 can illuminate the proximal end 212 of the tip section 210 to give an attending physician an indication of where the expandable capacity of the catheter begins. The band shown can be platinum strip or other noble metal with a relatively short length of between approximately 0.025-0.030 inches and a thin wall thickness (approximately 0.0005 inches) to minimize the impact on flexibility and the outer diameter of the catheter.

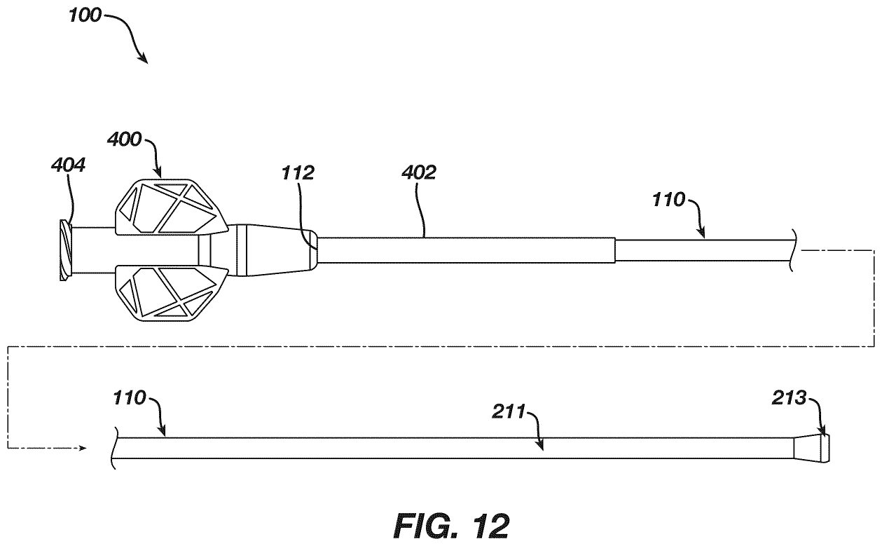

The braid of the tip section and shaft section can be formed monolithically as a single braid structure. In other examples, the band 118 can provide the linking structure for the proximal and distal portions of the catheter 100 between the proximal end 212 of the braid of the tip section 210 and the distal end of elongate shaft 110. This joint can allow different material and complex braid configurations to be used for the proximal and distal portions of the catheter and linked for a relatively low manufacturing cost and higher yields. It also allows for the braids of the proximal elongate shaft 110 and tip section to be quickly manufactured separately to any of a number of desired lengths. If the total catheter length is around 1350 to 1380 mm, the tip section 210 can be approximately 100 mm in length, leaving around 1250 mm of elongate shaft 110 terminating proximate a connection for the catheter (e.g., proximate a connection hub 400 as shown in FIG. 12). For more complex geometries or a more gradual stiffness transition, the tip section can be up to approximately 400 mm in length bonded with an 850 mm shaft at a more proximal marker band 118 location.

In another embodiment, DFT platinum-filled wires can be used for at least one of the distal tip braid wires to make the tip radiopaque under fluoroscopy. Alternatively, radiopaque coils or bands can be threaded around the atraumatic loops to provide visibility to the tip.

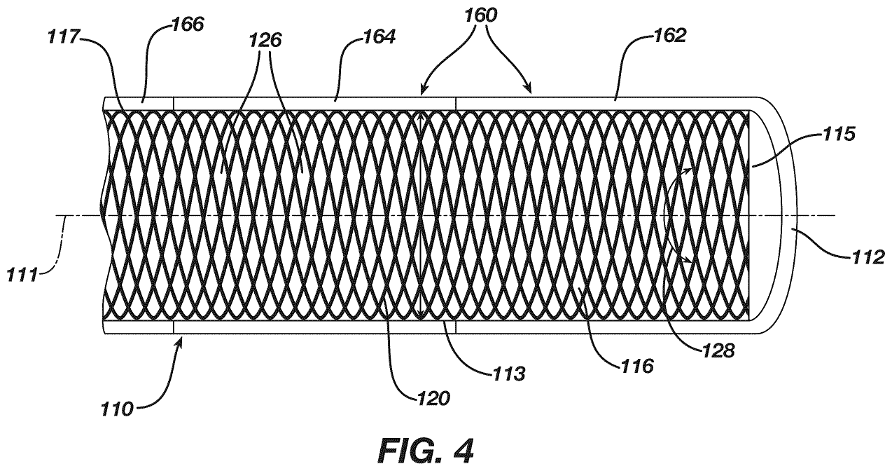

FIG. 4 depicts a cross section of the most proximal region of the elongate shaft 110. The plurality of braided segments 120 making up the support for the shaft can have a relatively dense weave with circumferentially elongated rings of cells 126 providing good scaffolding for the greater stiffness of the more proximal polymer body jackets 160. The braid segments 120 can be dimensionally stable such that the half the wires are wound in one direction (for example, clockwise) while the other half of the wires are wound in the opposing direction. The structure can have other supporting members, such as helical coils extending underneath the braid, to resist tensile elongation and offer improved kink resistance.

An inner liner 115 can provide a low friction inner surface without any sagging or other structural protrusions into the interior lumen 116 during aspiration. Alternately, wrapped ePTFE liners can be used to provide a low friction inner surface while also giving the structure high tensile strength to resist tensile elongation. In addition to or instead of the inner liner 115, hydrophilic coatings on one of both of the inner and outer surfaces of the shaft can also be used.

As mentioned, the elongate shaft 110 and tip section 210 can be sized to be compatible with relatively low-profile access sheaths and catheters, so that a puncture wound in the patient's groin (in the case of femoral access) can be easily and reliably closed. For example, the catheter 100 can be required to pass through the lumen of a sheath or guide with an inner diameter of less than 0.110 inches, preferably 0.090 inches, in some cases less than 0.087 inches, and most preferably less than 0.085 inches. Therefore, the catheter shaft can have an overall delivery profile with an inner diameter 113 of approximately 0.070-0.072 inches (0.084 inch or 2 mm outer diameter), and yet be able to expand its distal tip and mouth to the size of the vessel approximate where the clot is located, which could be as large as 5 mm.

The kink resistance of the shaft is in part due to the structure of the braided segments 120 in cooperation with the outer polymer body jackets 160. Adjusting the braid picks per inch (“PPI”, e.g., pick 223 shown in FIG. 10A), pitch, and cell angle, combined with pre-stretching or shortening other liner and/or outer jacket materials, can effectively set the longitudinal stiffness and the force required to bend different sections of the shaft. As will be appreciated, a “pick”, e.g., pick 223 in FIGS. 10A and 10B, are locations that two strands of the braid segments 120 overlap. The braided segments 120 of the proximal shaft can have a PPI in a range from approximately 120 to approximately 170. Torqueability and kink resistance can be gained by using stainless steel wires in a diamond two-over-two pattern where two wires side by side alternately pass under two wires, then over two other wires. A herringbone pattern, or flattened wires, can also be utilized for a lower profile cross section. For example, flat braids can reduce the profile while offering a similar stiffness to a round braid of the same cross sectional area. For braid-only shafts, a reduced PPI can be used to increase flexibility for approximately the most distal 5 mm to 20 mm of the shaft.

Alternately, for braided segments 120 with reinforcement (such as coils), a lower density (for example, 60 PPI for a 0.071″ ID shaft) braid can be maintained from there or varied. Coiled support can be stopped away from the start of the funnel taper of the expandable tip so the structures can hinge between the funnel taper start and the rest of the shaft.

The polymer body jackets 160 can transition distally to progressively softer materials in a stepwise fashion for added flexibility. By way of example and not limitation, the elongate shaft 110 shown can have a most proximal jacket 162 which can span a significant distance (approximately 1000 mm, e.g., from 800 mm to 1200 mm) from the proximal end 112 can include Pebax, TR 55, ML 21, Nylon 12, or similar material having a hardness of approximately 72 or 85 Shore D. The next, second more distal jacket 164 can be shorter than the proximal jacket 162, can span approximately 10 to 50 mm, and can be composed of Pebax or similar elastomer with a lesser hardness than the proximal jacket 162, for example around 63 Shore D. The next more distal jacket 166 can extend a further 20-60 mm and have a lesser hardness than the second more distal jacket 164, for example around 55 Shore D. It is appreciated that jacket lengths can be varied for the different sections to achieve a design that is optimal for most anatomies.

One or more axial spines 117, although not necessary for all disclosed embodiments, can be used as an additional link between the shaft body and the tip. The spine or spines 117 can counteract tensile elongation and contribute to the push characteristics of the shaft. This can be especially beneficial for the expandable tip as a large stiff clot can become lodged at the distal end of the catheter and can subject it to large tensile forces as the catheter is retracted into a larger outer sheath for removal from the vessel. The spine can be positioned beneath the braid, threaded between weaves of the braid, located on the outer diameter of the braid, or some combination of these. The spine can be composed of metallics, a polymeric, or composite strands such as Kevlar. In some preferred examples a liquid crystal polymer (LCP), such as Technora, can be utilized which is easy to process and offers high tensile strength without sacrificing any lateral flexibility.

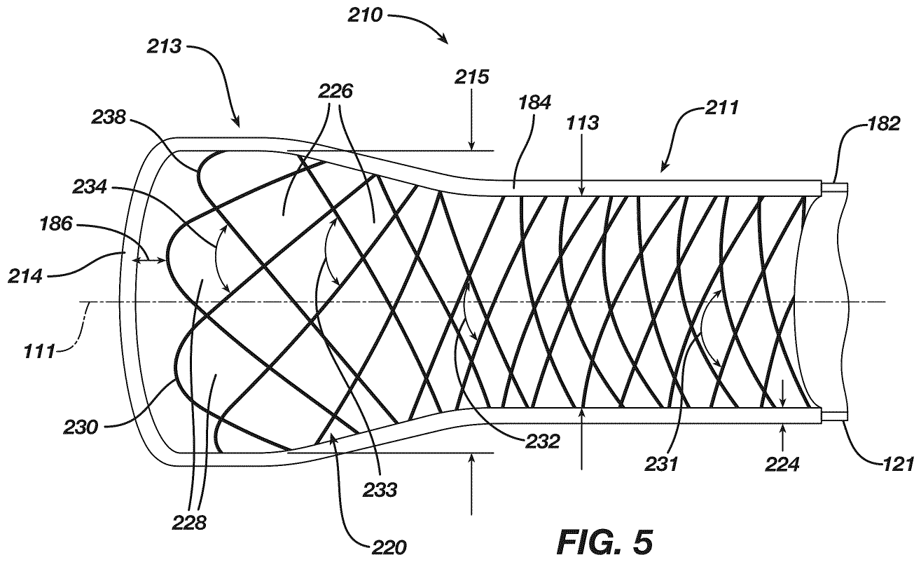

A magnified partial cross section view of an example internal configuration for the tip section 210, post-expansion, for the large bore catheter is depicted in FIG. 5. The more proximal tubular body 211 of the section can have approximately the same inner diameter 113 of the elongate shaft 110 while the more distal portion can be expanded to a larger inner diameter 215 at or near the distal end 214. The plurality of braids 220 can provide good scaffolding for the outer jackets 182, 184, especially in regions where a particularly soft and highly flexible polymer or blend with less structure is chosen.

The wires of the braided segments 220 can be Nitinol or another shape memory superelastic alloy so that the solid state phase transformations can be designed to dictate the constrained deliver and unconstrained deployed diameters of the tip. The braid can thus have an inner diameter 113 approximate that of the catheter bore when constrained and be heat set to a free shape with a larger inner diameter 215 for when the catheter 100 is deployed from the outer sheath. The wires can also be drawn filled tubing (DFT) shape memory alloy with a platinum core such that the braid is visible under fluoroscopy. A further benefit of using a superelastic material for the braid wires is that the catheter walls can be relatively thinner without sacrificing performance characteristics such as flexibility or crush strength, adding robustness to tortuous bends for the tip section 210.

The inner diameter 215 of the expanded tip 210 will vary based on the nominal diameter of the catheter. A catheter with an inner diameter 113 of approximately 0.070″ in the collapsed can have a tip section 210 with a maximum inner diameter 215 of approximately 0.090 inches in the expanded deployed configuration. Similarly, catheters with shafts in other common sizes, such as 5Fr up to 9Fr, can also be envisioned with flared tip diameters 215 which scale accordingly, for an overall range of approximately 0.075-0.200 inches.

As discussed previously, the braid wires can invert 238 proximally back upon themselves as shown to form distal hoops 230 at the distal end 214 of the tip braid sections 220. This forms the braid in a one over one half-diamond pattern where a single wire passes under, then over another single wire. Fewer wires can thus be used as each individual wire will form a hoop 230. The hoops eliminate any potential of braid ends migrating through the polymer encapsulation during use. Two or more wires can also be tied together as one for additional reinforcement in the braid.

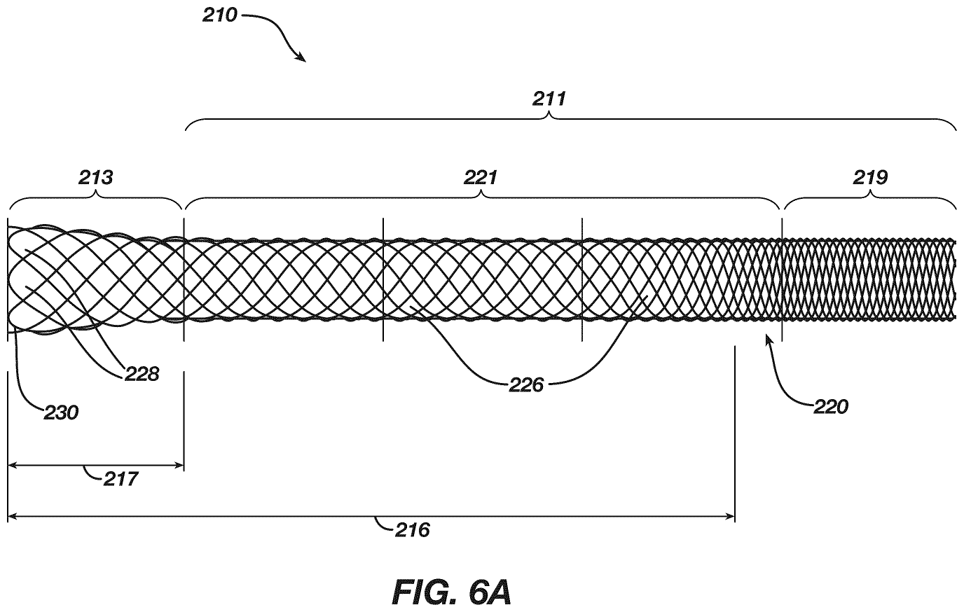

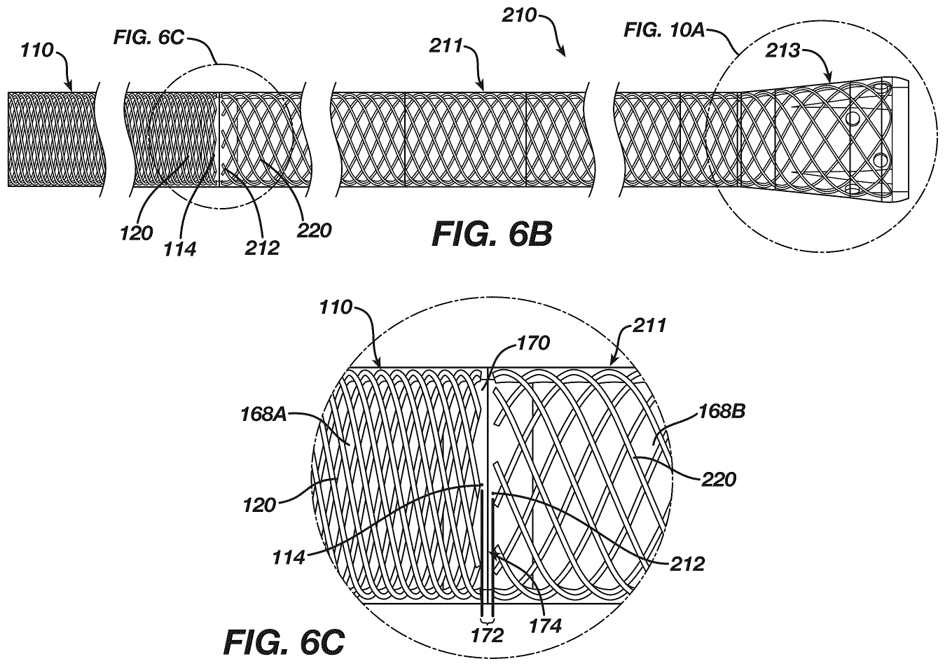

It is possible that the entire catheter supporting braid (i.e. both the elongate shaft 110 and tip section 210) can be a single monolithic braid of one material extending from the proximal end 112 to the distal end 214 with hoops 230. However, it may be difficult to manufacture the looped distal hoops 230 with a braid that is greater than 1200 mm in length with reasonable yield rates. It is feasible to manufacture a distal tip section 210 having braids 220 that are at most 400 mm long and join to simple proximal braid segments 120 of the elongate body. Nitinol braid wires like those of the tip can be used for the proximal braids, but less expensive stainless steel wires can perform in these regions for stiffness and with less cost. As will be described below with reference to FIG. 6C, the plurality of wire braided segments 120 of the elongate shaft 110 can be separated from the plurality of wire braided segments 220 of the distal dip section 210 via a transition 172.

The plurality of braids 220 making up the tip section 210 can form circumferential rings of closed cells 226. The cells 226 can deform as the braid wires slide relative to one another, and are capable of elongating circumferentially with respect to the longitudinal axis 111 when the distal expansile portion 213 is radially expanded to the deployed configuration, and elongating axially (flattening) with respect to the longitudinal axis when the expansile portion is reduced to the collapsed delivery configuration for advancement (or when the tip is withdrawn back into an outer sheath). The cells 226 can be substantially diamond shape to allow the vertices to facilitate more uniform deformation/expansion as shown or take some other profile if the braid wires follow a non-linear pattern.

The crossover of the braid wires form braid angles 231, 232, 233, 234 at the vertices of each of the cells 226, which help define the expansile capabilities of different portions of the tip. In addition to the heat set shape memory providing the funnel-shaped profile for the tip, variations in the cell angles allows for further expansion of the tip during clot aspiration. Generally, the braid angles can become smaller and more acute as the distal end 214 is approached to aid in allowing greater expansion for interacting with and receiving a clot. This is important because a clot must be deformed in order to fit into the catheter lumen, and firm clots with high coefficients of friction do not tend to deform and reshape easily and thus do not readily conform to the passive shape of the catheter tip. Reactive expansion can allow the catheter tip to effect a seal and consequent suction grip on the clot so that it can be retracted to safety.

Nominal angles can be chosen to balance the desired competing capabilities of the tip (delivery and target site performance). Smaller braid angles yield greater expansion capability to conform to the contours of a large or firm, fibrin-rich clot as it is ingested. This added expansion allows for better clot management and reduces the risk of shearing when compared to other tips with stiffer, less compliant frames or those utilizing stiffer polymeric materials. Further, smaller angles offer less resistance to collapse and better force transmission when transiting through an outer guide sheath. However, these characteristics can come at the cost of some flexibility and radial force to resist tip collapse during aspiration.

Alternately, high braid angles (where the wires are aligned more radially) can provide better compressive hoop strength in an expanded state to resist collapse of the tip under aspiration. More obtuse cell angles can also limit the ability of the expanded tip to over-expand radially in compression when the catheter 100 is being advanced through a vessel independently (after deployment from the guide sheath). This can help the expanded tip avoid snagging in vessels, particularly in tight bends and when being pushed through vessels of progressively smaller diameters. However, the radial force can complicate the collapse of large cell angles for delivery through and retraction into the guide sheath, the friction causing the catheter to bind or be otherwise undeliverable. Furthermore, the folding of large angles requires elongation of the expansile portion 213 of the tip which, if excessive, can potentially exceed the elastic limit of the distal tip jacket 184.

A distal marker band 121 approximately 0.8 mm in length can be included just proximal of the distalmost tip jacket 184 to give radiopacity near the distal end 214. The marker band 121 can be platinum or another suitable noble metal and can be crimped over the axial spine (not shown) and the braid of the tip section can extend over the band. The band 121 can also be situated at or near the distal end 119 of the inner liner 115 at least 5 mm and up to 10 mm from the distal end 214 of the tip section 210.

To be compatible with many of the most widely adopted guides and/or sheaths, the inner diameter 113 of the catheter elongate shaft 110 and the expanded inner diameter 215 of the expandable tip section 210 can be sized and scaled appropriately. For example, a 5Fr catheter targeting vessels approximately 2.0 mm in diameter can have a shaft inner diameter 113 of approximately 0.054 inches and an expanded tip inner diameter 215 in a range from approximately 0.068-0.090 inches. Similarly, a 6Fr catheter targeting vessels approximately 2.3-3.4 mm in diameter can have a shaft inner diameter 113 of approximately 0.068-0.074 inches and an expanded tip 210 inner diameter 215 in a range from approximately 0.090-0.120 inches. A larger 8Fr catheter for less remote clots can have a shaft inner diameter 113 of approximately 0.082-0.095 inches and an expanded tip 210 inner diameter 215 in a range from approximately 0.090-0.188 inches. The upper bounds of the expanded tip diameter 215 is limited by delivery forces when traversing within an outer guide or sheath. These common sizes can result in the ratio of the inner diameter 113 of the elongate shaft 110 to the maximum expanded inner diameter 215 of the flared tip 210 being in a range from approximately 0.55-0.90.

To create a more atraumatic vessel crossing profile, the distalmost tip jacket 184 can extend for a distance 186 to overhang beyond the distal hoops 230 of the tip section braided segments 220. The distance can be in a range of approximately 0.1-1.0 mm or, more preferably, 0.5-0.8 mm. The jacket 184 can be the softest of those on the catheter and can cover approximately the distal 100 mm of the length. In one example, polymer with a hardness of approximately 62 Shore A can be reflowed to form the distalmost tip jacket 184. In some cases, an even softer layer of approximately 42 Shore A can be used. As will be described in greater detail below with respect to FIG. 11, an additional overlap jacket 300 can also be added to provide a soft, atraumatic distal profile for the catheter 100.

The example illustrated in FIG. 6A shows how the plurality of braided segments 220 making up the tip section 210 can have variable PPI and braid angles. Sixteen Nitinol wires in the one over one half-diamond pattern can be used to produce eight distal hoops 230. More proximal sections can have a greater PPI than more distal regions. Generally, the PPI of the braid will have an inverse relationship with the expandability of a particular section of the tip 210. Regions with a larger PPI can have greater flexibility but limited expansion capability. Similarly, regions with lower PPI values can trade hoop strength for markedly greater expansion capability.

The braided segments 220 can sub-classified into segments by the braid characteristics and their contribution towards the expansion capabilities of that segment. The braid in a most proximal section 219 can be the “stiff” or “more proximal” section as described herein. An intermediate expansile portion 221 can alternately be desired as a “intermediate” or “mid” section. Similarly, the distal expansile portion 213 with the greatest radial expansion capability can be referred to as the “least stiff” or “more distal” section.

The distalmost cells 228 can have the smallest cell angle corresponding to the greatest expansion capability. FIG. 6A illustrates a final cell angle of 85 degrees, but designs having angles from 65 up to 125 degrees have been contemplated. The braid angles of the distal expansile portion 213 can increase over the length 217 of this section. The intermediate expansile portion 221 can have a tubular profile with a gradual progressive increase in cell angle and PPI (from approximately 40 up to 140) proximally to allow localized expansion as an ingested clot transits the section and is further compressed. This pattern can be maintained in the proximal section braid 219 for the remainder of the tip section 210.

Designs can have expansile portions with axial lengths 216 that are relatively long (for example, between approximately 5 mm and 10 mm) for the best clot management characteristics as a longer section can ingest and compress more of a clot. Alternatively, the length 216 can be kept short (for example, between approximately 1 mm and 5 mm) for improved hoop strength and trackability. Generally, it has been found that braid angles of 110 degrees or lower gives radial expansion under compression, with lower angles offering greater expansion capability. Therefore, the length 216 of the expansion zones can be tailored by changing the distances at which angles of 125 degrees or lower are maintained. Alternatively, the expansion zone can have a variable braid angle over a length.

Referring now to FIG. 6B, the side view of the catheter 100 shows transitions from the proximal elongate shaft 110 to the distal tip section 210, according to aspects of the present invention. As is described herein, the stiffness of the catheter 100 can transition from a stiffer construct proximally to a softer, more pliable, atraumatic material distally. The differing stiffness can be provided by the selecting appropriate polymer jackets over the braided segments 120, 220, because the length of the entire catheter can have plurality (e.g., five to eight) outer polymer jackets 160, 180 extending from the proximal end 112 of the elongate shaft 110 to the distal end 214 of the expansile portion 213. A hardness of a most proximal polymer body jacket 162 can be approximately 72 Shore D in hardness, and a hardness of a distalmost, or final tip jacket 184 can be approximately 42 Shore A in hardness. Extending from proximal to distal, the jackets 160, 180 can have a hardness of approximately 72 Shore D at most proximal polymer body jacket 162, to a second jacket having a hardness of approximately 63 Shore D, to a third jacket having a hardness of approximately 55 Shore D, to a fourth jacket having a hardness of approximately 45 Shore D, to a fifth jacket having a hardness of approximately 35 Shore D, to a sixth jacket having a hardness of approximately 73 Shore A, and to a seventh jacket (e.g., a final tip jacket 184) having a hardness of approximately 42 Shore A. As will be described below, the catheter 100 can include a distally positioned end profile jacket 300, which can have a hardness of approximately 42 Shore A or softer.

FIG. 6C is a detailed view of the area circled proximally in FIG. 6B, and the figure shows a transition from the proximal elongate shaft 110 to the distal tip section 210, according to aspects of the present invention. As described above, in certain implementations, the band 118 (see FIG. 3) can provide the linking structure for the proximal and distal portions of the catheter 100, for example between the proximal end 212 of the braid of the tip section 210 and the distal end 114 of elongate shaft 110. In other implementations, and as shown in FIG. 6C, the series of jackets can help link the first plurality of wire braided segments 120 and the second plurality of wire braided segments 220. FIG. 6C shows an example catheter 100 wherein the proximal elongate shaft 110 is separated from the distal tip section 210 via a transition 172. In some examples, the transition 172 can be a gap between the first (proximal) plurality of wire braided segments 120 and the second (distal) plurality of wire braided segments 220, wherein the two different braided segments 120, 220 do not contact each other. This can help to ensure the entire length of the catheter 100, which can be over 1200 mm in length, can be more easily manufactured, while it also allows the two different braided segments 120, 220 to be manufactured from different materials. In the instance that the transition 172 is a gap, the catheter 100 can include a transition jacket 170 positioned proximate the transition 172. The transition jacket 170 can have a hardness greater than a hardness of either of a first polymer jacket 168A proximal to the transition 172 and a second polymer jacket 168B distal to the transition 172. It will be appreciated that the first polymer jacket 168A or the second polymer jacket 168B cane be any of the jackets discussed herein, including for example outer jacket 182. To illustrate using a non-limiting example, the first polymer jacket 168A and the second polymer jacket 168B can both be relatively soft and pliable since they are positioned proximate the distal tip section 210. Therefore, the first polymer jacket 168A and the second polymer jacket 168B can have a hardness of approximately 42 Shore A, whereas the hardness of the transition jacket 170 can be approximately 45 Shore D.

Kink resistance should be considered at the transition 172. Hardness ranging from 45 Shore D to 72 Shore D have been employed for the transition jacket 170. Hardness of 55 Shore D to 72 Shore D can be implemented, but the kink diameter may be high (e.g., around >5 mm). Hardness of 45 Shore D can provide an adequate kink diameter (e.g., <5 mm). That said, a hardness of 35 Shore D and 73 Shore A can be implemented for the transition jacket as well. The first polymer jacket 168A, the second polymer jacket 168B, and the transition jacket 170 can all be at 42 Shore A also, but this may cause a kink at the transition 172.

Regarding the materials of the two different braided segments 120, 220, one aspect of the present disclosure provides different materials on either side of the transition 172. One aspect of the present disclosure is to provide a catheter 100 that marries good pushability proximally and good flexibility distally. As such, the transition jacket 170 can help to bridge a first (proximal) plurality of wire braided segments 120 having a harder/stiffer material than the second (distal) plurality of wire braided segments 220. The first plurality of wire braided segments 120 of the elongate shaft 110 can include stainless steel, for example a high strength stainless steel as 304V steel. The second distal plurality of wire braided segments 220 of the distal tip section 210 can be softer and more complaint so as to more easily traverse the vasculature while also providing preferred visibility under fluoroscopy. The second distal plurality of wire braided segments 220 can comprise, for example, Nitinol or a composite of Nitinol with a tantalum or platinum core. These materials also provide a degree of shape memory so as to help set the expanded deployed configuration for the expansile portion 213. The second distal plurality of wire braided segments 220 can additionally or alternatively comprise tungsten. In some examples, there may be no transition 170, and in these examples, there is just one material for the braid 120, 220 across its entire length. This material can be stainless, tungsten, Nitinol, or a Nitinol composite as described herein. Another example includes a mixture of two different types of materials along the length of the one or more braids 120, 220. For example, the one or more braids 120, 220 can include a first quantity of wires of a first material and a second quantity of wires of a second material, all intermixed along the length of the catheter 100. One embodiment can include a combination of stainless steel and tungsten, with eight stainless steel wires (with four distal hoops 230) and eight tungsten wires (with four distal hoops 230). This example would reduce the cost of tungsten if using for the full length of the catheter 100 (e.g., no joint or transition 172).

Both the tungsten and Nitinol composites described above are much more radiopaque than the stainless steel and allow an operator to see the second distal plurality of wire braided segments 220 under fluoroscopy, especially where the braid density is high. This allows the operator to visualize the shape of the expansile portion 213 of the catheter 100 while trying to access difficult locations and further allows the physician to control the catheter 100 more precisely. In the case that the material of the expansile portion 213 is not sufficiently radiopaque to be able to see the expansile portion 213, for example if the braid density is lower, then the radiopaque pucks 176 described herein can be employed. Tungsten is more radiopaque than stainless steel and can reduce the number of radiopaque pucks 176 needed to visualize the distal end 214 of the expansile portion 213 under fluoroscopy. For example, an expansile portion 213 made of Nitinol with a core may benefit from eight radiopaque pucks 176, whereas an expansile portion 213 made of tungsten may only need four radiopaque pucks 176. In one embodiment, the catheter 100 can employ stainless steel with >425KSI for the first (proximal) plurality of wire braided segments 120 and tungsten with >400KSI for the second (distal) plurality of wire braided segments 220, both having >1% elastic strain. The catheter 100 can therefore rely on the elastomeric properties of the one or more jackets 160, 180 to recover the shape after collapse through a sheath for delivery and subsequent deployment. It is also conceived to employ the use of stainless steel with 325KSI proximally (120) and tungsten with >600KSI distally (220), the greater strength of tungsten distally can improve kink resistance where there are softer jackets 190 or potentially lower braid PPI. As will be appreciated, higher PPI can provide better kink resistance while lower PPI improves flexibility.

The transition between a harder braid segment 120 proximally and a more complaint braid segment 220 distally, in conjunction with the transition from harder polymer body jackets 160 proximally to softer polymer distal jackets 180 distally, help to provide improved pushability for the catheter 100 while also maintaining flexibility distally.

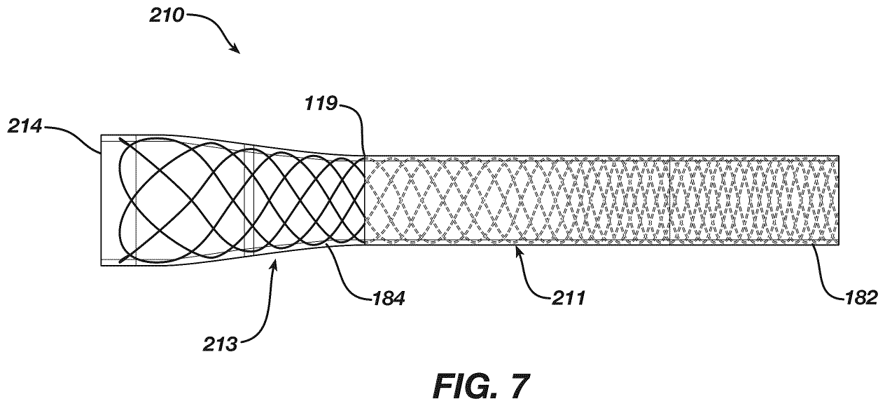

The braided segments 120, 220 can be positioned between the aforementioned inner liner 115 and the outer jackets 160, 180, and as such the combination of the liner and jackets can keep the braided segments 120, 220 from separating at the transition 172. The liner 115 can be a wrapped PTFE liner that consists of (i) two pieces of tape wrapped in a helix in one direction with (ii) two pieces of tape side-by-side wrapped over the first layer in an opposite helix direction, otherwise known as WEPL. The WEPL does not stretch until around >20N load and provides the tensile properties needed for the transition 172. This is in comparison to solution cast PTFE that stretches at around 1N and extruded PTFE that stretches at around 10N. It is contemplated that stretch resistance up to more than 10N, or more than 20N, is preferred for the liner 115. In some examples, the junctions between the transition jacket 170 and the more proximal jacket 168A and more distal jacket 168B can be secured via an adhesive 174. The adhesive 174 can be limited to a minimum length so as to not obstruct flexibility at the transition 172. This minimum length can be less than 2 mm, and preferably less than 1 mm. The braid of the tip section 210 in FIG. 6A is shown with outer jackets 182, 184 applied in FIG. 7. The inner liner 115 can terminate some distance proximal of the distal end 214 of the tip section 210. The final tip jacket 184 can cover the distal 50-80 mm (e.g., approximately 60) of the tip section 210. This jacket 184 can be relatively soft, having a hardness of, for example and not limitation, around 42 Shore A. The next proximal outer jacket 182 can be approximately 80-120 mm (e.g., approximately 100 mm) in length and be Pebax with a hardness around 73 Shore A. It is appreciated that jacket lengths can be adjusted to provide a catheter with optimal transitions for particular anatomical destinations and are not limited to the lengths discussed in this application. Additional details about the transition of hardness from proximal to distal end of the catheter 100 is provided below.

In some examples, to allow for smooth delivery of the clot retrieval catheter through an outer catheter, the outer surface of the outer jackets 182, 184 can be coated with a low-friction or lubricious material, such as PTFE or commercially available lubricious coatings such as offered by Surmodics, Harland, Biocoat or Covalon. Similarly, the inner surface of the catheter lumen can also be coated with the same or similar low-friction material for the passage of auxiliary devices and to aid in a captured clot being drawing proximally through the catheter with aspiration and/or a clot retrieval device.

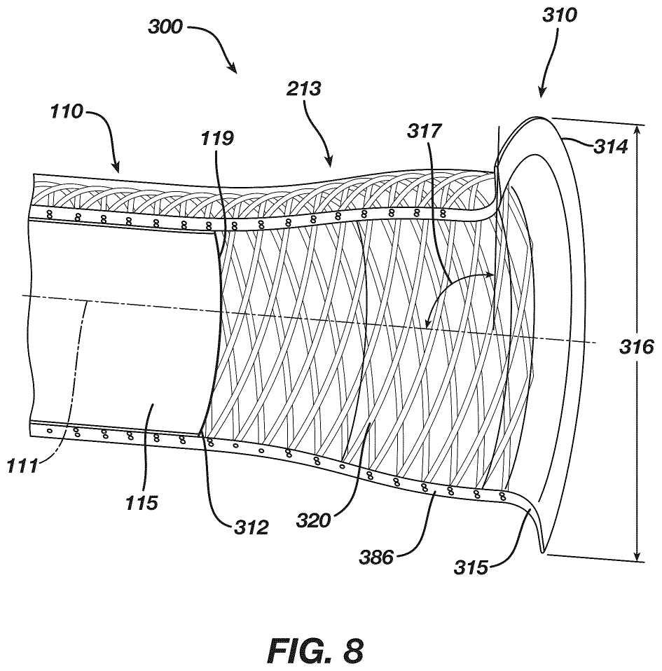

Referring now to FIG. 8, a flared polymer disk extension 315 can be provided at the large end of the distal expansile portion 213 of the tip to further increase the outer diameter of the flared braid structure. This provides a large aspiration mouth while also giving a flexible seal lip to interface with the walls of a vessel. The disk can be thin and flexible with shape memory properties, such as with elastomeric materials. The braided structure 320 gives the expansile portion 213 sufficient radial force to withstand the forces of aspiration while the unsupported disk extension 315 will have atraumatic properties to gently contact and seal with the vessel walls. The disk extension allows the outer diameter of the device to seal in large vessels while minimizing the volume of polymer-encapsulated braid that needs to be collapsed through an outer guide sheath for delivery and removal from a vessel. Although disk extension 315 can form an angle 317 perpendicular to the longitudinal axis 111 as manufactured, the angle can vary in a substantial range. For example, the unsupported disk can invert proximally to allow the catheter to be advanced distally within the vessel without causing trauma. In other examples, the disk extension can lean proximally or distally.

The disk extension 315 can be formed through molding or by reflowing the polymer over a flared mandrel that includes a step profile to shape the lip. The profile can be wrapped or compressed in the distal direction prior to loading into an outer guide sheath. Once advanced from the guide sheath and deployed, the disk can pop and contact the vessel walls as the catheter is advanced into progressively narrower paths. Another advantage of such a design is that the disk extension 315 can easily be collapsed for delivery, but the sharp angle formed on deployment means the disk will not have a tendency to be vacuumed into the mouth of the catheter during aspiration which could otherwise cause an obstruction. The disk can also be provided in various diameters 316 that can be tailored to perform in set ranges of vessel diameters. For example, a 3 mm outer diameter disk can be tailored for vessels with an inner diameter of 2 to 3 mm. Similarly, a 7 mm outer diameter disk can be tailored for 4 to 7 mm diameter vessels.

It should be noted that any of the herein disclosed catheters designs can also be used with one or more stentrievers. The combined stentriever retraction and efficient aspiration through the enlarged tip section in the expanded deployed configuration can act together to increase the likelihood of first pass success in removing a clot. The catheter can also direct the aspiration vacuum to the clot face while the stentriever holds a composite clot (comprised of friable regions and fibrin rich regions) together preventing embolization and aid in dislodging the clot from the vessel wall. The funnel-like shape of the tip section can also reduce clot shearing upon entry to the catheter and arrest flow to protect distal vessels from new territory embolization.

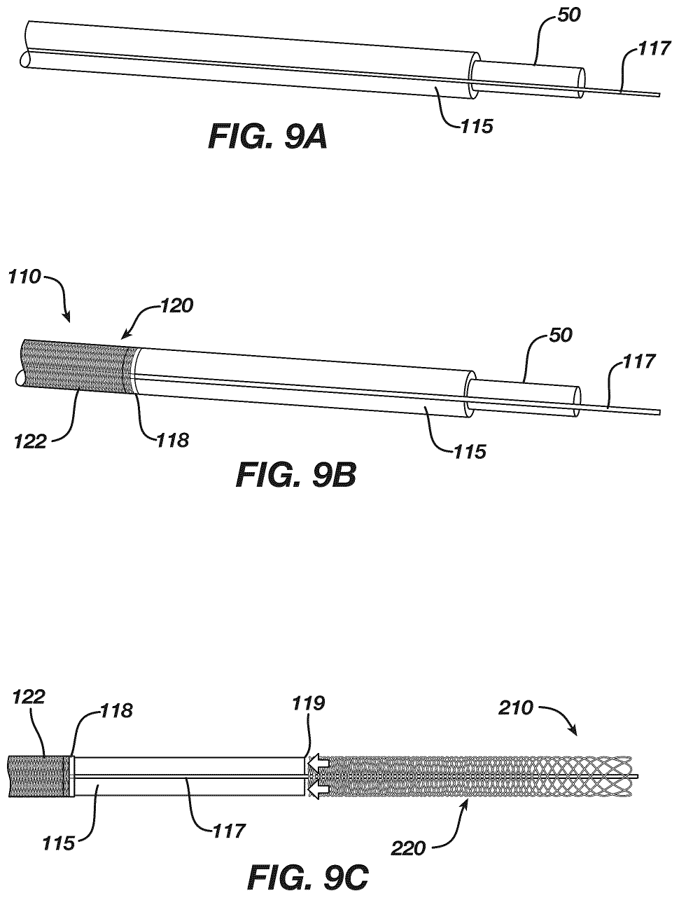

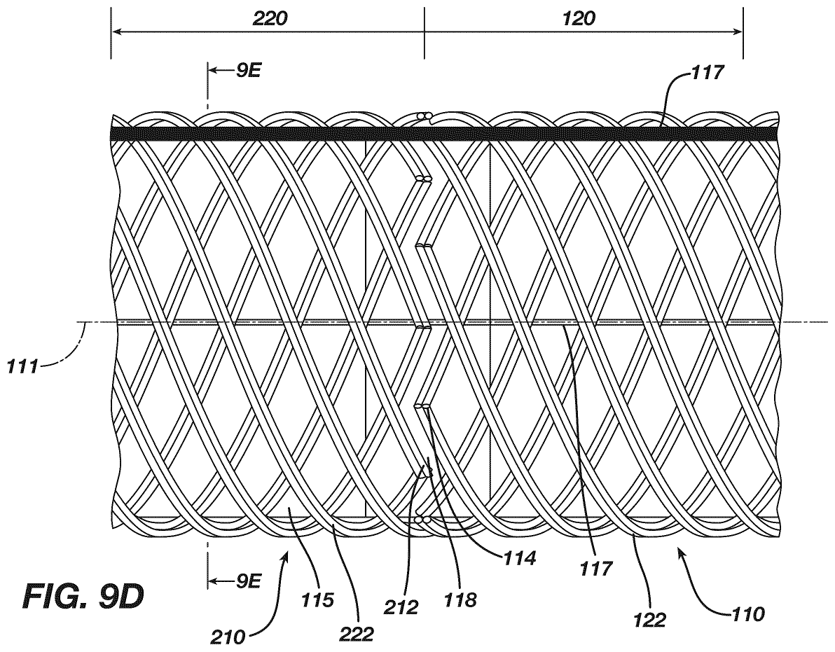

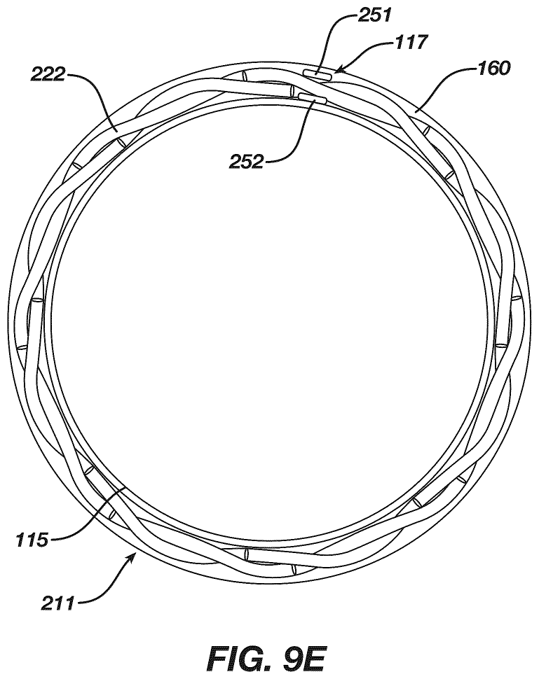

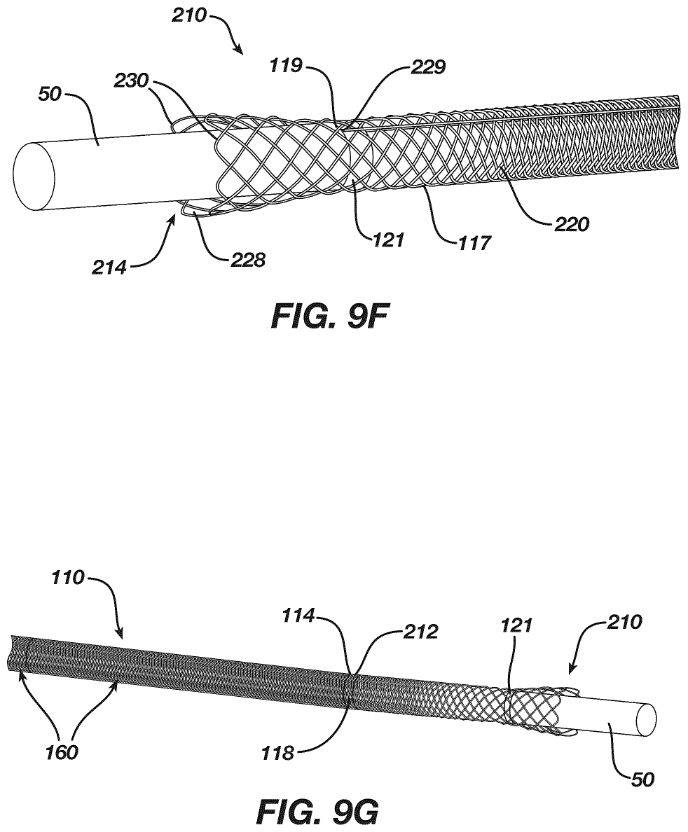

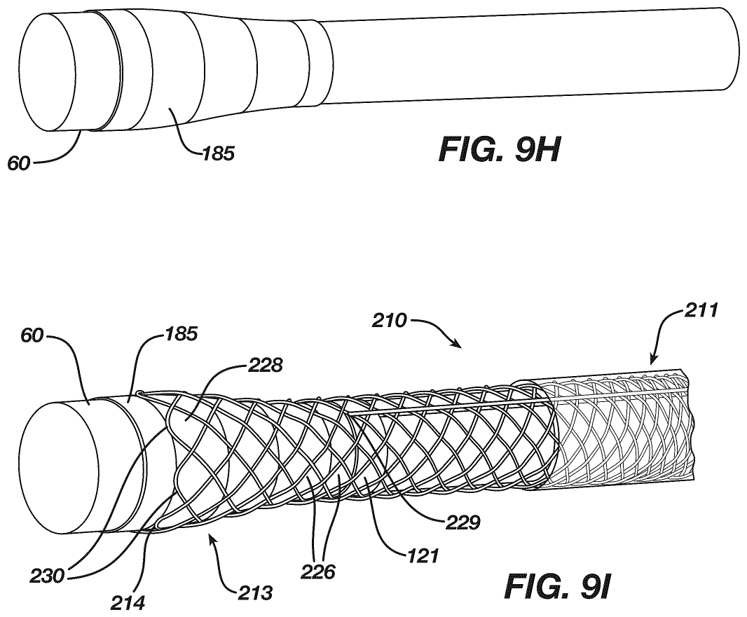

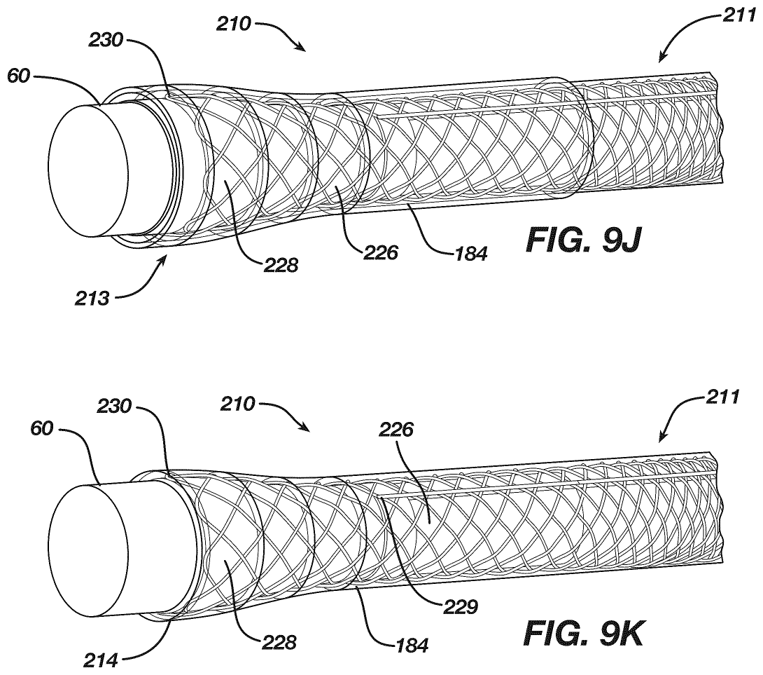

A method for manufacturing a catheter utilizing the disclosed designs is graphically illustrated in FIGS. 9A-9K. FIG. 9A shows a low friction inner liner 115 positioned on a supporting application mandrel 50. The mandrel can often be silver-plated copper (SPC) as is commonly used for these applications. Alternatively, especially ductile materials (such as PEEK) can be used which stretch to neck down in diameter so that the mandrel can be removed after completion of the catheter assembly. Further mandrel materials can include nylon coated copper or nylon coated steel.

The inner liner 115 can be a lubricious, low friction material such as film cast PTFE with a very low wall thickness (0.00075 inches or more preferably 0.0003 inches) and include thin outer tie layers to help with bonding. The liner can aid in a clot being pulled proximally through the catheter with aspiration and/or a clot retrieval device such as a stentriever. The liner can also help with the initial delivery of any associated devices to the target site through the lumen of the catheter. Depending on the material chosen, it can also be stretched to alter the directionality of the liner material (e.g. if the liner material has fibers, an imposed stretch can change a nominally isotropic sleeve into a more anisotropic, longitudinally oriented composition) to reduce the wall thickness as required. The liner can alternatively be of a wrapped ePTFE structure with high tensile strength to improve the integrity of the assembly and reducing tensile elongation to prevent delamination of the liner while maintaining adequate lateral flexibility.

In another embodiment, an elastomeric liner may be used that is impregnated with low friction fillers, which can enhance lateral flexibility and allow the shaft to stretch to a degree without plastically deforming. The stretch can reduce the likelihood of delamination as the liner will expand with the elastomeric outer jackets as the shaft is subjected to tensile loads and also as the shaft navigates tortuous bends (when in tortuous bends, the inner bend radius defined by the catheter is in compression while the outer bend radius of the catheter is in tension). Multiple liners may also be utilized to achieve different properties, for example, a liner with high tensile strength may be used for the majority of the shaft and span across the joint between the proximal and distal braids, while the distal end of the shaft near the funnel tip may use an elastomeric liner to improve lateral flexibility.

An axial spine 117 can be positioned along the tie layer or above an etched liner surface beneath the braid to counteract any tensile elongation of the shaft. The spine can be of a threaded formulation such as Zylon or another LCP so that it is not stiff in compression and can be flattened beneath the braid for a reduced cross sectional profile. A liquid crystal polymer can offer the highest tensile strength, while a stainless steel spine or Nitinol can offer the best pushability at the cost of some lateral flexibility. As another option, some of the LCP spine can be replaced with a stainless steel and/or Nitinol spine. In a further embodiment, Aramid fibers can be used as the spine.

Initially, a length of the spine can extend distally well beyond the distal ends of the inner liner and application mandrel to aid with further assembly steps during manufacturing. Other examples can use additional spines, such as two spines spaced 180 degrees apart, for added tensile strength.

An elongate body support shaft 110 with a braided segment 120 is positioned around at least a proximal portion of the inner liner 115 and application mandrel 50 in FIG. 9B. The braids can extend distally from a luer on the proximal end of the catheter to overlap with a radiopaque proximal marker band 118 positioned proximal to the distal end of the inner liner 115. A distal marker band (see FIG. 9F) can also be crimped into place at or near the distal end of the inner liner before or after application of the spine.

A braided tip section 210 is threaded proximally over the application mandrel 50 and inner liner 115 using the distal portion of the spine 117 as a guide, as illustrated in FIG. 9C. In some examples, the tip section 210 can be formed from wires of Nitinol or another shape memory superelastic alloy so that the solid state phase transformations can be designed to dictate the unconstrained size of the tip when it is deployed from the distal end of a guide sheath. The tip section can have at least a proximal tubular portion with a nominal inner diameter roughly the same size as the elongate body, but the braid can be compressed to expand over the liner and overlap with the marker band 118 during assembly. Alternatively, the braid can be made to have a minimum clearance of 0.0005″ over the diameter of the max liner OD.

The distalmost braid pattern 122 of the plurality of braid segments 120 around the elongate shaft 110 can overlap with the marker band 118 at their distal end 114. Similarly, the most proximal braid pattern 222 of the tip section braided segments 220 can overlap at their proximal end 212 with the band 118. Both overlapping braids can be disposed over the spine 117 and welded to the marker band 118, as depicted in FIG. 9D. In some instances, the braided sections can be woven together for additional strength prior to being welded. Alternately, the braided sections could be welded together directly, or incased in a more proximal (approximately 200 mm to 300 mm proximal of the tip) polymer jacket of high tensile strength. This jacket can be, for example, Pebax 25D or up to Pebax 72D depending on the desired final stiffness of the catheter. The joint may also be strengthened by placing it over liners with high tensile strength, such as Junkosha wrapped ePTFE liners.

Though the proximal braid segment 120 of the elongate shaft 110 can also be produced from Nitinol, the use of a mid-joint for the braids at the marker band 118 allows it to be manufactured from stainless steel for increased axial stiffness and reduced cost. This gives the advantage of allowing more complex design features in the distal Nitinol braid of the tip section 210 to join with lower cost standard proximal braid and/or coil sections at the marker band 118, as seen in the longitudinal section in FIG. 9D and the cross section in FIG. 9E. This flexibility increases manufacturing yields through the separation of complex and standard catheter shaft sections. The use of specific metallics such as platinum (which can be welded to both stainless steel and Nitinol) for the sleeve of the marker band 118 can replace the use of adhesives or other means and create a more robust joint. The band can be kept relatively short, for example between 0.3-1.0 mm, in order to minimize the impact on shaft flexibility.

The axial thread of the spine 117 can be pulled through a distal braid opening of the tip section 210 and looped proximally to connect the spine more securely to the distal region of the catheter. The loop 229 can create both an outboard spine portion 251 extending atop the braid pattern 222 and an inboard spine portion 252 running beneath the braid (see FIG. 9E). The loop can be positioned more proximally, such as at the distal end 119 of the inner liner 115 as seen in FIG. 9F. Alternatively, the loop 229 can be at a more distal location up to or through the distalmost cells 228 of the tip section 210. The distance for which the spine 117 is looped back on itself can be kept short (<5 mm) or can be a much greater distance of approximately 50 mm. In many examples, after inverting at the loop 229 the spine 117 can return all the way to the proximal end of the catheter shaft. In another embodiment, the spine need only extend as far as the joint between the proximal and distal braids.

FIG. 9G shows that once the spine has been looped back, an axial series of separate outer polymer body jackets 160 can be reflowed or laminated in place on the elongate shaft 110. The applied heat can allow the outer polymer to fill the interstitial sites between the braid of the elongate body and around the spine 117. This flow can also help to fix the jackets axially so they cannot slide distally. Depending on their axial location on the elongate shaft 110, the polymer body jackets 160 can alternately be sprayed, dipped, electro spun, and/or plasma deposited.

Suitable jacket materials can include thermoplastic elastomers like those of the Pebax family which can have a wide range of mechanical and dynamic properties. The polymer body jackets 160 can have differing durometer hardness and/or wall thicknesses. For example, a stiffer more proximal portion of the shaft can have a jacket with a thickness of approximately 0.004 inches and a hardness of around 72D. By contrast, a distal section requiring greater flexibility but where the underlying support braid is less dense can have a jacket with a wall thickness in the region of 0.003 inches and a hardness of around 40A. The jacket thickness can be optimized to fill the space between the liner and the braid to achieve a minimum wall thickness above the braid of 0.0005 inches to ensure there is no exposed braid.

FIG. 9H depicts a flared mandrel 60 with a Neusoft inner liner 185 applied over at least the stepped portion of the mandrel. In FIG. 9I, the application mandrel 50 can be removed and the flared mandrel 60 with the inner jacket 185 loaded proximally into the distal end 214 of the assembly. A soft, elastic outer polymer tip jacket 184 extrusion can be threaded or stretched over the flared mandrel tool 60 and pushed over the flared section to expand the undersized material of the extrusion (see FIG. 9J). Alternatively, the jacket 184 material can be sized to fit over the stepped portion of the mandrel 60. FIG. 9K illustrates the outer jacket 184 and inner jacket 185 reflowed to the tip section.

In some instances, a temporary sleeve of PTFE/FEP or other suitable low friction material can be applied over the reflow mandrel and used to control and arrest the distal flow of the distal tip jacket 184 as it is reflowed into place. Typically, a layer of heat shrink can be positioned around the jacket 184 extrusion to better conform the material to the contours of the stepped mandrel during reflow or lamination. The heat shrink pushes the melted material into the braid and does not stick to the jackets allowing removal after reflow. Once complete, flared mandrel 60 can be removed from the assembly, and if necessary, any excess material can be trimmed away to ensure the desired catheter profile is attained. The trimmed tip may then be subjected to a tipping process to apply a chamfer or fillet to the jacket material for a more atraumatic finish. When removed at least the distal outer 20 cm of the distal tip section of the catheter can be coated with a hydrophilic coating.

FIG. 10A is a detailed view of the area circled distally in FIG. 6B, and the figure shows a distal tip section 210 of a catheter 100, according to aspects of the present invention. The figure shows how the expansile portion 213 can be defined by a number of closed cells 226 formed by overlapping strands of the braided segments 220. These closed cells 226 can form circumferential rings of cells, including for example the rings labeled as the first circumferential rings of cells 226A, the second circumferential rings of cells 226B, and the third circumferential rings of cells 226C. The labeled first circumferential rings of cells 226A is the most proximal ring of the expansile portion 213, the second circumferential rings of cells 226B is the next more distal ring, and the third circumferential rings of cells 226C is located proximate a distal end 214 of the expansile portion 213 and comprises the distalmost cells 228 of the expansile portion 213. The expansile portion 213 can include from five (5) to nine (9) circumferential rings of cells 226 between the proximal tubular body 211 and the distal end 214 of the expansile portion 213. The examples shown in FIGS. 10A and 10B each have seven rings, identified by labels D1-D7 in the figures, where D8 is the first ring within the tubular proximal body 211 and D1 is the distalmost ring (i.e., distal circumferential rings of cells 226C). Each closed cell 226 is defined both proximally and distally by a pick 223, as defined above. As such, an expansile portion 213 with seven rings D1-D7 can have eight picks 223, identified by labels P1-P7 in FIGS. 10A and 10B (P1 defining the distalmost cell 228 and P8 being the first pick at the transition to the expansile portion 213). This arrangement of circumferential rings can help to maintain the expanded deployed configuration. More details about the arrangement of the cells 226 within the circumferential rings are provided with reference to FIG. 10B below.

The circumferential rings can also provide a location for one or more radiopaque markers, which can help a physician locate a distal end 214 of the expansile portion 213 under fluoroscopy. As shown, the expansile portion 213 can include one or more radiopaque pucks 176. The radiopaque pucks can be cylindrical, as shown, but can include any other shape. The cylindrical, or rounded, shape provides an atraumatic profile and can help to ensure the liner 115 and/or jacket(s) 180 are not torn or pierced by the puck. The radiopaque pucks 176 can comprise a dense radiopaque material including, for example, platinum iridium. In one example, one or more radiopaque pucks 176 can be positioned within individual cells of the distalmost cells 228 and within the distal circumferential rings of cells 226C, as shown in both FIGS. 10A and 10B. The catheter 100 can include two evenly spaced radiopaque pucks 176, three evenly spaced radiopaque pucks 176, four evenly spaced radiopaque pucks 176, or more pucks within that distal circumferential rings of cells 226C. Additionally or alternatively, one or more radiopaque pucks 176 can be positioned within a more proximal circumferential rings of cells, as shown in FIG. 10A. To illustrate, the expansile portion 213 can include a first quantity of radiopaque pucks 176 in the first circumferential rings of cells defined by the distalmost cells 228, and can include a second quantity of radiopaque pucks 176 in a more proximal circumferential rings of cells, and the first quantity and second quantity of radiopaque pucks 176 can be evenly spaced around the circumference of the expansile portion 213 to provide a good and consistent view of the device under fluoroscopy.

It can be beneficial to reduce the number of radiopaque pucks 176 and to keep them close together instead of spreading them around the expansile portion 213 (see for example FIG. 10B) to improve visibility. To do so, and as described above, the expansile portion 213 can be manufactured out of a (i) tungsten, (ii) Nitinol, or (iii) a composite of Nitinol with a tantalum or platinum core. This can help to reduce the manufacturing time and complexity. However, using a less dense material, for example a Nitinol braid with filler (e.g., tantalum or platinum), the expansile portion 213 may need additional radiopaque pucks 176 for proper visibility, for example eight pucks. Adding eight radiopaque pucks 176 can be completed by increasing the wall thickness of the radiopaque pucks 176 from WT0.003£ to 0.004″ or 0.005″. Another option is to increase marker density at the distal end 214 of the expansile portion 213. One could employ eight evenly spaced radiopaque pucks 176 or more, such as twelve to twenty radiopaque pucks 176. These additional radiopaque pucks 176 could be placed at the most distal circumferential rings of cells 226C, or some could be placed in the next two to three adjacent and more proximal circumferential rings of cells 226.

As described above, the braided segments 120, 220 can be positioned between the aforementioned liner 115 and the outer jackets 160, 180. As such, the radiopaque pucks 176 can be positioned between the inner liner 115 and the distal tip jacket 184, and within a closed cell 226. If the radiopaque pucks 176 are between the liner 115 and distal tip jacket 184, a glue or adhesive may be necessary to hold the two layers together during the liner during assembly. Alternatively, the radiopaque pucks 176 can be positioned between a first inner layer of polymer jacket 180 and a second outer layer of polymer jacket 180. The inner jacket of the two layers can be reflowed first, then it is slit to insert a radiopaque puck 176 before placing the outer layer and then reflowing to melt the inner and outer jackets together and fully encapsulate the radiopaque pucks 176. In this example, a glue may not be required.

Referring again to the liner 115, as described above, preferred liner materials can include PTFE, wrapped ePTFE, or similar materials. To enable the inner liner 115 to expand with the expansion of the expansile portion 213, the distal end 119 of the liner 115 can have one or more slits 178. In some examples, the distal end can include two slits 178 spaced evenly around the circumference of the liner (see FIG. 10B). In other examples, more slits 178 can be placed, for example three slits 178, four slits (see FIG. 10A; only the two foreground slits are visible since the two background slits align with the foreground slits), or more. The slits 178 can extend proximally entirely to the first pick 223 of the expansile portion 213 (marked P7), or the slits 178 can extend to an intermediate position along the length of the expansile portion 213, such as to the fourth pick 223 (marked P4) from the distal end 214.

Referring specifically to FIG. 10B for illustration, and as mentioned above, the crossover of the braid wires can form braid angles 231, 232, 233, 234 at the vertices (e.g., picks 223) of each of the cells 226, and these braid angles 231, 232, 233, 234 can define the expansile capabilities of different portions of the tip. The transition of braid angles 231, 232, 233, 234 (which could also be defined through pick 223 width) at the expansile portion 213 can be one of the keys in allowing the expansile portion 213 to self-collapse for delivery through a sheath while also having enough radial force to resist collapse during aspiration. It has been found that the identified five (5) to (9) circumferential rings help to ensure the self-expansion and collapse. Further, the braid angles 231, 232, 233, 234 can also decrease gradually from proximal to distal to provide an even transition and to prevent the expansile portion 213 from locking up during forward advancement to collapse into a sheath. However, it can be challenging to define manufacturing tolerances that enable this benefit. Accordingly, the tolerances of the transition from one circumferential ring of cells 226 to the next can be such that a first (more proximal) braid angle 231 is greater than or equal to a second (more distal) braid angle 232 minus 5°. Stated otherwise, D7>D6−5°, D6>D5−5°, D5>D4−5°, D4>D3−5° and so on. Further, the first braid angle 231 of the expansile portion 213 can be approximately 65°, and the most distal braid angle 234 can be approximately 53°. For example, the first braid angle 231 can be from approximately 60° to approximately 70°, and the most distal braid angle 234 can be from approximately 48° to approximately 58°.

FIG. 11 is a detailed view of the area circled in FIG. 10B and shows a distal tip section 210 of a catheter 100 with a distal overlap jacket 300, according to aspects of the present invention. One aspect of the present disclosure is to provide a catheter 100 with an atraumatic distal end. In some examples, the distal end 214 of the expansile portion 213 can include a distal overlap jacket 300 that includes a contoured edge 302. The jackets 180 can be reflowed, and the distal overlap jacket 300 can be assembled by adding heat shrink material over the distal end 214 and then applying heat to melt the jackets 180 and shrink the heat shrink material at the same time so the distal overlap jacket 300 is reflowed to bond with other polymer distal jackets 180 and adhere to the liner 115. The distal overlap jacket 300 can be molded onto the distalmost tip jacket 184, as shown. The distal end 304 of the distal overlap jacket 300 can extend from the distal end 214 of the expansile portion 213 a certain distance 306. The hardness of the distal overlap jacket 300 can be similar to the hardness of the distalmost tip jacket 184. For example, if the hardness of the distalmost tip jacket 184 is 42 Shore A, then the hardness of the distal overlap jacket 300 can be 42 Shore A. It is also contemplated that the distal overlap jacket 300 can be softer or harder than the distalmost tip jacket 184. There can be a benefit in using harder materials at the distal overlap jacket 300 to maintain the shape of the expansile tip 213 and prevent it from being over-compliant, and as such the hardness can be in the range of 42 Shore A to 4 Shore 5 D, or preferably 73 Shore A to 35 Shore D.

FIG. 12 illustrates a catheter 100 attached to a connection hub 400, according to aspects of the present invention. The catheter 100 can be a system including a catheter connection hub 400 connected to the proximal end 112 of the elongate shaft 110. The connection hub 400 has a strain relief extension 402 extending therefrom. The connection hub 400 has a proximal locking mechanism 404 configured to engage with a known aspiration device. The strain relief extension 402 is a length of polymeric tubing that extends from within the connection hub 400 to a position distal to the hub 400 and over a length of the proximate elongate shaft 110 to prevent the proximate elongate shaft 110 from crimping or breaking near the connection hub 400. The strain relief extension 402 can have a hardness of approximately 45-65 Shore D, for example 55 Shore D. The strain relief extension 402 can extend distally over the elongate shaft 110 for a length of approximately 30 mm, or for example anywhere from 15-20 mm, 20-25 mm, 25-30 mm, 30-35 mm, 35-40 mm, etc. The proximal locking mechanism 404 can be a threaded connection or any other type of connection to connect the catheter 100 to an auxiliary device.

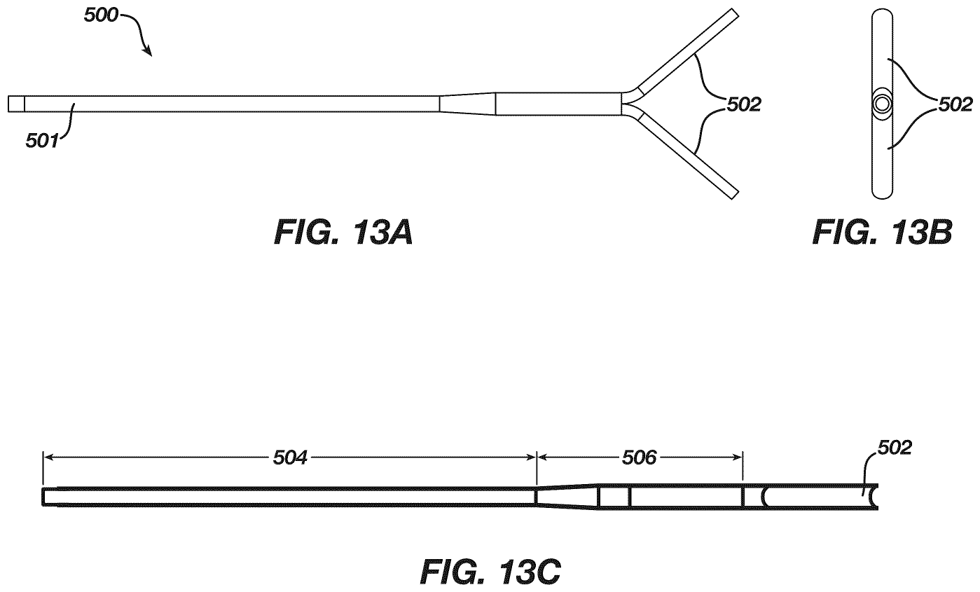

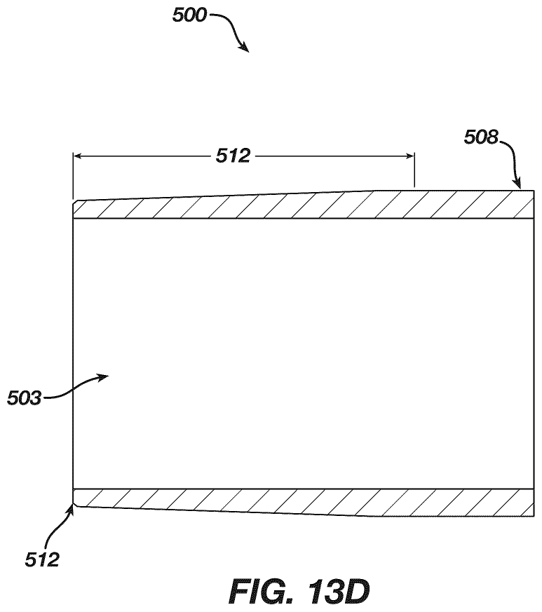

FIGS. 13A-13D illustrate an example insertion tool 500. FIG. 13A provides a side view of the insertion tool 500, FIG. 13B provides an end view of the insertion tool 500, FIG. 13C provides a top elevational view of the insertion tool 500, and FIG. 13D provides a detail view of a distal tip 508 of the insertion tool 500, according to aspects of the present invention. As can be seen, the insertion tool 500 includes an elongated shaft 501 with a lumen 503 into which the catheter 100 can be inserted. The insertion tool 500 can include low-friction polymer, such as PTFE. High density polyethylene (HDPE), low density polyethylene (LDPE), and/or Pebax 72D are also alternative materials for the insertion tool 500 and also have low friction. In some examples, the insertion tool 500 can include a hydrophilic coating in the inner diameter (ID) of the tool so as to have low friction and allow easy passage of the expansile portion 213 therethrough. In a preferred embodiment, although not required, the insertion tool 500 is clear for visualization of the expansile portion 213 during insertion. Alternatively, so as to not inadvertently lose the insertion tool 500 if it is transparent, the tool can have spiral colored stripes along the length of the insertion tool 500.

In some examples, the of the insertion tool 500 includes one or more pull tabs 502 extending from its proximal end. These pull tabs can be manufactured of a tearable material, such as a peelable PTFE material. The insertion tool 500 further includes a distal shaft section 504 and a proximal flare section 506. The larger ID of the proximal flare section 506 is sized to allow unrestricted passage of the diameter (OD) of the expansile portion 213 so that there is no resistance and the expansile portion 213 is easy to insert the funnel. The length of this proximal flare section 506 is long enough to provide support for the expansile portion 213 during advancement into the distal shaft section 504 with smaller a ID. The distal shaft section 504 is sized to be similar to the ID of the guide catheter to allow easy passage of the catheter 100 from insertion tool 500 to guide catheter.

Turning now to FIG. 13D, the figure shows a distal tip 508 of the insertion tool 500. The distal tip 508 has a tapered OD along a tapered portion 510 to allow it to insert further into the tapered lure of a guide catheter to ensure the ID of the insertion tool 500 is as close as possible to the ID of the guide catheter. This can prevent the expansile portion 213 from deploying in the space between the insertion tool 500 and guide catheter, which can cause the expansile portion 213 to lock up during insertion. There is also a rounded distal end 512 that reduces abrasion if the expansile portion 213 is accidentally pushed distal of the distal tip 508 and then retracted back through the distal tip 508 for insertion. If the distal tip 508 is a simple cut edge, it is sharp and can scrape or strip any hydrophilic coating from the distal end 508 of the expansile portion 213, which will increase friction and cause the expansile portion 213 to lock up inside the guide catheter or insertion tool 500.

Examples of the present disclosure can be implemented by any of the following numbered clauses:
Clause 1: A catheter (100) comprising: a proximal elongate shaft (110) comprising a distal end (114), a lumen (116), and a first plurality of wire braided segments (120); a distal tip section (210) connected to the distal end (114) of the proximal elongate shaft (110), the distal tip section (210) comprising (i) a proximal tubular body (211), (ii) an expansile portion (213) having a collapsed delivery configuration and an expanded deployed configuration, and (iii) a second plurality of wire braided segments (220) comprising circumferential rings of cells (226), the distal end (214) of the expansile portion (213) comprising distal hoops (230) where wires of the second plurality of wire braided segments (220) invert to loop back through the second plurality of wire braided segments (220) and create distalmost cells (228) of the expansile portion (213); one or more polymer body jackets (160) disposed around the proximal elongate shaft (110) and one or more polymer distal jackets (180) disposed around the distal tip section (210); and one or more radiopaque pucks (176) positioned within one or more of the distalmost cells (228).
Clause 2: The catheter (100) of Clause 1 comprising at least two radiopaque pucks (176) positioned within a first circumferential rings of cells (226) defined by the distalmost cells (228).
Clause 3: The catheter (100) of Clause 1 comprising at least four radiopaque pucks (176) positioned within a first circumferential rings of cells (226) defined by the distalmost cells (228), the four radiopaque pucks (176) evenly positioned around the first circumferential rings of cells (226).
Clause 4: The catheter (100) of Clause 1 wherein a first radiopaque puck (176A) of the one or more radiopaque pucks (176) is positioned in one of the distalmost cells (228), and wherein the catheter (100) comprises a second radiopaque puck (176B) positioned in a first circumferential rings of cells (226) proximal to the distalmost cells (228).
Clause 5: The catheter (100) of Clause 1 further comprising an inner liner (115) positioned internally to the first plurality of wire braided segments (120) and the second plurality of wire braided segments (220), the inner liner (115) comprising at least one longitudinal slit (178) at its distal end (119) to enable expansion of the expansile portion (213) into the expanded deployed configuration.
Clause 6: The catheter (100) of Clause 5 further comprising at least two longitudinal slits (178) at the distal end (119) of the inner liner (115). a
Clause 7: The catheter (100) of Clause 5, wherein the one or more radiopaque pucks (176) are positioned between a polymer distal jackets (180) and the inner liner (115).
Clause 8: The catheter (100) of Clause 5, wherein the one or more radiopaque pucks (176) are positioned between an inner layer and an outer layer of the polymer distal jackets (180).
Clause 9: The catheter (100) of Clause 1, wherein the expansile portion (213) comprises between 6 and 8 circumferential rings of cells (226) between the proximal tubular body (211) and the distal end (214) of the expansile portion (213).
Clause 10: The catheter (100) of Clause 1, wherein: the expansile portion (213) comprises at least a first circumferential rings of cells (226A) proximate a transition between the proximal tubular body (211) and the expansile portion (213), a second circumferential rings of cells (226B) adjacent and distal to the first circumferential rings of cells (226A), and a third circumferential rings of cells (226C) proximate a distal end (214) of the expansile portion (213); the first circumferential rings of cells (226A) comprises closed cells (226) having a first braid angle (231), the second circumferential rings of cells (226B) comprises closed cells (226) having a second braid angle (232), and the third circumferential rings of cells (226C) comprises closed cells (226) having a third braid angle (234); and the first braid angle (231) is approximately 65°, and the third braid angle (234) is approximately 53°.
Clause 11: The catheter (100) of Clause 1, wherein the one or more polymer body jackets (160) and the one or more polymer distal jackets (180) transition from a harder material to a softer material, wherein a hardness of a most proximal polymer body jacket (162) is approximately 72 Shore D in hardness, and wherein a hardness of a distalmost end profile jacket (302) is approximately 42 Shore A in hardness.
Clause 12: The catheter (100) of Clause 1, wherein the proximal elongate shaft (110) comprises stainless steel, and the distal tip section (210) comprises at least one of (i) tungsten, (ii) Nitinol, or (iii) a composite of Nitinol with a tantalum or platinum core.
Clause 13: The catheter (100) of Clause 1, wherein the first plurality of wire braided segments (120) and the second plurality of wire braided segments (220) comprise the same strands of wires.
Clause 14: A catheter (100) comprising: a proximal elongate shaft (110) comprising a distal end (114), a lumen (116), and a first plurality of wire braided segments (120); a distal tip section (210) connected to the distal end (114) of the proximal elongate shaft (110), the distal tip section (210) comprising (i) a proximal tubular body (211), (ii) an expansile portion (213) having a collapsed delivery configuration and an expanded deployed configuration, and (iii) a second plurality of wire braided segments (220) comprising circumferential rings of cells (226); a plurality of polymer jackets (160,180) extending from the proximal elongate shaft (110) to proximate the distal end (214) of the expansile portion (213), the plurality of polymer jackets (160,180) comprising a first polymer jacket (168A) and a second polymer jacket (168B); and a transition jacket (170) positioned proximate a transition (172) between the first plurality of wire braided segments (120) and the second plurality of wire braided segments (220), the transition jacket (170) having a hardness greater than a hardness of either of the first polymer jacket (168A) proximal to the transition (172) and the second polymer jacket (168B) distal to the transition (172).
Clause 15: The catheter (100) of Clause 14, wherein a stiffness of the plurality of polymer jackets (160,180) ranges from approximately 75 Shore D harness proximally to 40 Shore A hardness distally.
Clause 16: The catheter (100) of Clause 14, wherein the transition (172) is a gap between the first plurality of wire braided segments (120) and the second plurality of wire braided segments (220).
Clause 17: The catheter (100) of Clause 16 further comprising an inner liner (115) positioned internally to the first plurality of wire braided segments (120) and the second plurality of wire braided segments (220), wherein the first plurality of wire braided segments (120) and the second plurality of wire braided segments (220) are adhered to the inner liner (115) and the transition jacket (170) proximate the transition (172).
Clause 18: The catheter (100) of Clause 17 further comprising at least two longitudinal slits (178) at the distal end (119) of the inner liner (115).
Clause 19: The catheter (100) of Clause 14, wherein the expansile portion (213) comprises from 5 to 9 circumferential rings of cells (226) between the proximal tubular body (211) and the distal end (214) of the expansile portion (213).
Clause 20: The catheter (100) of Clause 14, wherein the distal end (214) of the expansile portion (213) comprises distal hoops (230) where wires of the second plurality of wire braided segments (220) invert to loop back through the second plurality of wire braided segments (220) and create distalmost cells (228) of the expansile portion (213).
Clause 21: The catheter (100) of Clause 20, further comprising one or more radiopaque pucks (176) positioned within one or more of the distalmost cells (228).
Clause 22: The catheter (100) of Clause 14, wherein the proximal elongate shaft (110) comprises stainless steel, and the distal tip section (210) comprises at least one of (i) tungsten, (ii) Nitinol, or (iii) a composite of Nitinol with a tantalum or platinum core.
Clause 23. The catheter (100) of Clause 14, wherein the second plurality of wire braided segments (220) has a higher radiopacity than the first plurality of wire braided segments (120). 24: The catheter (100) of Clause 14, wherein at least one of the first plurality of wire braided segments (120) or the second plurality of wire braided segments (220) comprises wire braids of two different materials.
Clause 25: The catheter (100) of Clause 24, wherein the two different materials comprise two materials selected from the group consisting of stainless steel, tungsten, Nitinol, and a composite of Nitinol with a tantalum or platinum core.
Clause 26: A catheter (100) comprising: a proximal elongate shaft (110) comprising a distal end (114), a lumen (116), and a first plurality of wire braided segments (120); a distal tip section (210) connected to the distal end (114) of the proximal elongate shaft (110), the distal tip section (210) comprising (i) a proximal tubular body (211), (ii) an expansile portion (213) having a collapsed delivery configuration and an expanded deployed configuration, and (iii) a second plurality of wire braided segments (220) comprising circumferential rings of cells (226); and one or more polymer body jackets (160) disposed around the proximal elongate shaft (110) and one or more polymer distal jackets (180) disposed around the distal tip section (210), wherein a distal end (214) of the expansile portion (213) comprising distal hoops (230) where wires of the second plurality of wire braided segments (220) invert to loop back through the second plurality of wire braided segments (220), and wherein the expansile portion (213) comprises at least a first circumferential rings of cells (226A) proximate a transition between the proximal tubular body (211) and the expansile portion (213), a second circumferential rings of cells (226B) adjacent and distal to the first circumferential rings of cells (226A), and a third circumferential rings of cells (226C) proximate a distal end (214) of the expansile portion (213), wherein the first circumferential rings of cells (226A) comprises closed cells (226) having a first braid angle (231), the second circumferential rings of cells (226B) comprises closed cells (226) having a second braid angle (232), and the third circumferential rings of cells (226C) comprises closed cells (226) having a third braid angle (234), and wherein the first braid angle (231) is greater than or equal to the second braid angle (232) minus 5°, the first braid angle (231) is approximately 65°, and the third braid angle (234) is approximately 53°.
Clause 27: The catheter (100) of Clause 26, wherein the distal end (214) of the expansile portion (213) comprises distal hoops (230) where wires of the second plurality of wire braided segments (220) invert to loop back through the second plurality of wire braided segments (220) and create distalmost cells (228) of the expansile portion (213).
Clause 28: The catheter (100) of Clause 27 comprising at least two radiopaque pucks (176) positioned within a first circumferential rings of cells (226) defined by the distalmost cells (228).
Clause 29: The catheter (100) of Clause 28 comprising at least four radiopaque pucks (176) positioned within a first circumferential rings of cells (226) defined by the distalmost cells (228), the four radiopaque pucks (176) evenly positioned around the first circumferential rings of cells (226).
Clause 30: The catheter (100) of Clause 28 or 29 comprising a first radiopaque puck (176A) positioned in one of the distalmost cells (228), and a second radiopaque puck (176B) positioned in a first circumferential rings of cells (226) adjacent and proximal to the distalmost cells (228).
Clause 31: The catheter (100) of Clause 26 further comprising an inner liner (115) positioned internally to the first plurality of wire braided segments (120) and the second plurality of wire braided segments (220), the inner liner (115) comprising at least one longitudinal slit (178) at its distal end (119) to enable expansion of the expansile portion (213) into the expanded deployed configuration.
Clause 32: The catheter (100) of Clause 31 further comprising at least two longitudinal slits (178) at the distal end (119) of the inner liner (115).
Clause 33: The catheter (100) of Clause 26, wherein the expansile portion (213) comprises between 6 and 8 circumferential rings of cells (226) between the proximal tubular body (211) and the distal end (214) of the expansile portion (213).
Clause 34: The catheter (100) of Clause 26, wherein the one or more polymer body jackets (160) and the one or more polymer distal jackets (180) transition from a harder material to a softer material, wherein a hardness of a most proximal polymer body jacket (162) is approximately 72 Shore D in hardness, and wherein a hardness of a distalmost end profile jacket (302) is approximately 42 Shore A in hardness.
Clause 35: The catheter (100) of Clause 26, wherein the proximal elongate shaft (110) comprises stainless steel, and the distal tip section (210) comprises at least one of (i) tungsten, (ii) Nitinol, or (iii) a composite of Nitinol with a tantalum or platinum core.
Clause 36: An insertion tool (500) for a catheter (100) comprising: an elongated shaft (501) with an inner lumen (503), the elongated shaft (501) comprising: a distal shaft section (504); a proximal flare section (506) having an inner diameter greater than that of the distal shaft section (504); and a distal tip (508) having a tapered portion (510).
Clause 37: The insertion tool (500) of Clause 36 further comprising one or more pull tabs 502 extending from a proximal end of the proximal flare section (506).
Clause 38: The insertion tool (500) of Clause 36, wherein the distal tip (508) comprises a rounded distal end (512) configured to reduce abrasion of an expansile portion (213) of a catheter (100).
Clause 39: The insertion tool (500) of Clause 36, wherein a transition between the proximal flare section (506) and the distal shaft section (504) is smooth so as to provide support for an expansile portion (213) of a catheter (100) during advancement of the expansile portion (213) into the distal shaft section (504).
Clause 40: The insertion tool (500) of Clause 36, wherein the inner lumen (503) comprises a hydrophilic coating.

The invention is not necessarily limited to the examples described, which can be varied in construction and detail. The terms “distal” and “proximal” are used throughout the preceding description and are meant to refer to a positions and directions relative to a treating physician. As such, “distal” or distally” refer to a position distant to or a direction away from the physician. Similarly, “proximal” or “proximally” refer to a position near or a direction towards the physician. Furthermore, the singular forms “a,” “an,” and “the” include plural referents unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 70.001% to 109.999%.

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

What is claimed is:

1. A catheter comprising:
a proximal elongate shaft comprising a distal end, a lumen, and a first plurality of wire braided segments;
a distal tip section connected to the distal end of the proximal elongate shaft, the distal tip section comprising (i) a proximal tubular body, (ii) an expansile portion having a collapsed delivery configuration and an expanded deployed configuration, and (iii) a second plurality of wire braided segments comprising circumferential rings of cells, the distal end of the expansile portion comprising distal hoops where wires of the second plurality of wire braided segments invert to loop back through the second plurality of wire braided segments and create distalmost cells of the expansile portion;
one or more polymer body jackets disposed around the proximal elongate shaft and one or more polymer distal jackets disposed around the distal tip section; and
one or more radiopaque pucks positioned within one or more of the distalmost cells.

2. The catheter of claim 1 comprising at least two radiopaque pucks positioned within a first circumferential rings of cells defined by the distalmost cells.

3. The catheter of claim 1 comprising at least four radiopaque pucks positioned within a first circumferential rings of cells defined by the distalmost cells, the four radiopaque pucks evenly positioned around the first circumferential rings of cells.

4. The catheter of claim 1 wherein a first radiopaque puck of the one or more radiopaque pucks is positioned in one of the distalmost cells, and wherein the catheter comprises a second radiopaque puck positioned in a first circumferential rings of cells proximal to the distalmost cells.

5. The catheter of claim 1 further comprising an inner liner positioned internally to the first plurality of wire braided segments and the second plurality of wire braided segments, the inner liner comprising at least one longitudinal slit at its distal end to enable expansion of the expansile portion into the expanded deployed configuration.

6. The catheter of claim 5 further comprising at least two longitudinal slits at the distal end of the inner liner.

7. The catheter of claim 5, wherein the one or more radiopaque pucks are positioned between a polymer distal jackets and the inner liner.

8. The catheter of claim 5, wherein the one or more radiopaque pucks are positioned between an inner layer and an outer layer of the polymer distal jackets.

9. The catheter of claim 1, wherein the expansile portion comprises between 6 and 8 circumferential rings of cells between the proximal tubular body and the distal end of the expansile portion.

10. The catheter of claim 1, wherein:
the expansile portion comprises at least a first circumferential rings of cells proximate a transition between the proximal tubular body and the expansile portion, a second circumferential rings of cells adjacent and distal to the first circumferential rings of cells, and a third circumferential rings of cells proximate a distal end of the expansile portion;
the first circumferential rings of cells comprises closed cells having a first braid angle, the second circumferential rings of cells comprises closed cells having a second braid angle, and the third circumferential rings of cells comprises closed cells having a third braid angle; and
the first braid angle is approximately 65°, and the third braid angle is approximately 53°.

11. The catheter of claim 1, wherein the one or more polymer body jackets and the one or more polymer distal jackets transition from a harder material to a softer material, wherein a hardness of a most proximal polymer body jacket is approximately 72 Shore D in hardness, and wherein a hardness of a distalmost end profile jacket is approximately 42 Shore A in hardness.

12. The catheter of claim 1, wherein the proximal elongate shaft comprises stainless steel, and the distal tip section comprises at least one of (i) tungsten, (ii) Nitinol, or (iii) a composite of Nitinol with a tantalum or platinum core.

13. A catheter comprising:
a proximal elongate shaft comprising a distal end, a lumen, and a first plurality of wire braided segments;
a distal tip section connected to the distal end of the proximal elongate shaft, the distal tip section comprising (i) a proximal tubular body, (ii) an expansile portion having a collapsed delivery configuration and an expanded deployed configuration, and (iii) a second plurality of wire braided segments comprising circumferential rings of cells;
a plurality of polymer jackets extending from the proximal elongate shaft to proximate the distal end of the expansile portion, the plurality of polymer jackets comprising a first polymer jacket and a second polymer jacket; and
a transition jacket positioned proximate a transition between the first plurality of wire braided segments and the second plurality of wire braided segments, the transition jacket having a hardness greater than a hardness of either of the first polymer jacket proximal to the transition and the second polymer jacket distal to the transition.

14. The catheter of claim 13, wherein a stiffness of the plurality of polymer jackets ranges from approximately 75 Shore D harness proximally to 40 Shore A hardness distally.

15. The catheter of claim 13, wherein the transition is a gap between the first plurality of wire braided segments and the second plurality of wire braided segments.

16. The catheter of claim 15 further comprising an inner liner positioned internally to the first plurality of wire braided segments and the second plurality of wire braided segments, wherein the first plurality of wire braided segments and the second plurality of wire braided segments are adhered to the inner liner and the transition jacket proximate the transition.

17. The catheter of claim 16 further comprising at least two longitudinal slits at the distal end of the inner liner.

18. The catheter of claim 13, wherein the second plurality of wire braided segments has a higher radiopacity than the first plurality of wire braided segments.

19. A catheter comprising:
a proximal elongate shaft comprising a distal end, a lumen, and a first plurality of wire braided segments;
a distal tip section connected to the distal end of the proximal elongate shaft, the distal tip section comprising (i) a proximal tubular body, (ii) an expansile portion having a collapsed delivery configuration and an expanded deployed configuration, and (iii) a second plurality of wire braided segments comprising circumferential rings of cells; and
one or more polymer body jackets disposed around the proximal elongate shaft and one or more polymer distal jackets disposed around the distal tip section,
wherein a distal end of the expansile portion comprising distal hoops where wires of the second plurality of wire braided segments invert to loop back through the second plurality of wire braided segments, and
wherein the expansile portion comprises at least a first circumferential rings of cells proximate a transition between the proximal tubular body and the expansile portion, a second circumferential rings of cells adjacent and distal to the first circumferential rings of cells, and a third circumferential rings of cells proximate a distal end of the expansile portion,
wherein the first circumferential rings of cells comprises closed cells having a first braid angle, the second circumferential rings of cells comprises closed cells having a second braid angle, and the third circumferential rings of cells comprises closed cells having a third braid angle, and
wherein the first braid angle is greater than or equal to the second braid angle minus 5°, the first braid angle is approximately 65°, and the third braid angle is approximately 53°.

20. The catheter of claim 19, wherein:
the distal end of the expansile portion comprises distal hoops where wires of the second plurality of wire braided segments invert to loop back through the second plurality of wire braided segments and create distalmost cells of the expansile portion; and
the catheter comprises at least two radiopaque pucks positioned within a first circumferential rings of cells defined by the distalmost cells.
